(19) United States
(12) Patent Application Publication
(10) Pub. No.: US 2026/0265671 A1
(43) Pub. Date: Sep. 10, 2026

(54) Automatic Data Amplification, Scaling and Transfer

(71) Applicant: AMGEN INC., Thousand Oaks, CA (US)

(72) Inventor: Aditya Tulsyan, Atlanta, GA (US)

(21) Appl. No.: 18/877,820

(22) PCT Filed: Jun. 29, 2023
(86) PCT No.: PCT/US2023/026517

§ 371 Date: Dec. 20, 2024

Related U.S. Application Data
(60) Provisional application No. 63/357,303, filed on Jun. 30, 2022.

(51) Int. Cl. C12M 1/36; G05B 13/04

(52) U.S. Cl. CPC C12M 41/48; G05B 13/042

(57) ABSTRACT

In a method for scaling data across different processes, first time-series data indicative of one or more input, state, and/or output parameters of a first process over time, and second time-series data indicative of one or more input, state, and/or output parameters of a second process over time, are obtained. The method also includes generating a scaling model specifying time-varying relationships between the input, state, and/or output parameters of the first and second processes, and transferring, using the scaling model, source time-series data associated with a source process to target time-series data associated with a target process. The source time-series data is indicative of one or more input, state, and/or output parameters of the source process over time, and the target time-series data is indicative of input, state, and/or output parameters of the target process over time. The method further includes storing the target time-series data in memory.

### FIELD OF DISCLOSURE

The present invention relates generally to the application of machine learning methods to automate and streamline the data transfer process between different processes, such as processes associated with different manufacturing sites, different products, and/or different scales.

### BACKGROUND

Despite decades of research and advancements in industrial process monitoring and control, existing monitoring methods are not particularly effective for use in batch processes, especially in the biopharmaceutical industry. Unlike other batch processes, biopharmaceutical processes pose a unique challenge from the process monitoring and control perspective, which can be referred to as the “Low-N problem.” The Low-N problem represents the situation where production history is limited, with “N” referring to the length of the production history or the number of historical campaigns for a drug product. The Low-N problem has its roots in the way any contemporary biopharmaceutical manufacturing company operates. In biopharmaceutical manufacturing, a drug product with a long production history often has a huge repository of historical campaign data to build robust monitoring models. However, as newer drugs are discovered and pushed into the market, a long production history is often not available. In fact, it is common for the production history to have only a few or even no historical campaigns before the actual GMP (good manufacturing practice) campaign. Real-time multivariate statistical process monitoring (RT-MSPM) for these manufacturing processes traditionally requires large, at-scale datasets to build representative models, which has limited its utility for critical operations associated with NPIs (new product introductions).

The Low-N problem manifest itself, among other places, in scale-up studies. A scale-up study typically involves attempts to replicate a laboratory process at successively larger stages in order to develop expectations of performance and a set of best practices for the ultimate industrial facility. The problem of finding scaling between variables is not a new problem, and has been extensively studied (particularly in scale-up studies) using the similitude theory. See Skoglund, 1967, Similitude: Theory and Applications, International Textbook Co.; see also Coutinho et al., 2016, Engineering Structures, 119:81-94. Similitude theory is a branch of engineering concerned with establishing the necessary and sufficient conditions of similarity among phenomena. See Coutinho et al., 2016, Engineering Structures, 119:81-94. A prototype model is said to have similitude with the real application if the two share geometric similarity, kinematic similarity, and dynamic similarity. Similitude theory is the primary theory behind many formulas in fluid mechanics, and is also closely related to dimensional analyses. See Sonin, 2001, “The Physical Basis of Dimensional analysis,” 2^{nd} ed., Massachusetts Institute of Technology; see also Yunus and Cimbala, 2006, “Fluid Mechanics: Fundamentals and Applications,” International Edition, McGraw Hill Publication. Similitude theory is widely used in hydraulic engineering to design and test fluid flow conditions in actual experiments using prototype models.

For example, the scale-up for the growth of microorganisms is based on maintaining a constant dissolved oxygen concentration in the liquid (broth), independent of bioreactor size. This is typically achieved by keeping the speed of the end (tip) of the impeller the same in both the pilot reactor and the commercial reactor. If the impeller speed is too rapid, movement of the impeller can lyse the bacteria. If the speed is too slow, the bioreactor contents will not mix well. Similitude theory can be used to calculate the required impeller speed in the commercial bioreactor given the speed in the pilot bioreactor. If x∈ [symbol] and y∈ [symbol] represent the rotational speeds (rpm) of impellers in the pilot and commercial bioreactors, respectively, then under geometric similarity and constant tip speed assumptions one can derive:

$$y = β x . \qquad ( Equation 1 )$$

where β=b₁/b₂, and b₁, b₂∈ [symbol] are the diameters of the impellers in the pilot and commercial-scale bioreactors, respectively. See Hubbard et al., 1988, Chemical Engineering Progress, 84:55-61. Given β and x, it is straightforward to calculate the impeller speed in the commercial bioreactor. Similar relationships between variables can also be discovered using kinematic and dynamic similarities. Note that similitude theory yields precise scaling models between variables using first-principles knowledge. Moreover, the scaling parameters are readily computable as a function of key process attributes or dimensionless numbers, such as Reynolds or Froude number. While the similitude theory provides scaling models between variables in scale-up studies, it suffers from several limitations: (a) the models are nontrivial to derive in complex studies, as they require a thorough understanding of the underlying process; (b) it is not always possible in practice to validate geometric, kinematic, and dynamic similarity; (c) the scaling parameters are often functions of process parameters/attributes or dimensionless numbers, which may not be directly measured or observed; (d) the scaling relationship does not account for known or unknown disturbances that may affect the signals (e.g., if a motor fault develops in the commercial-scale bioreactor, causing the impeller to rotate at a higher or lower speed, then the relationship in Equation 1 is no longer valid); and (e) while the similitude theory yields scaling models in scale-up studies, in other applications similitude-based scaling models might be difficult to derive.

Therefore, there exists a need for a general framework to determine scaling between any arbitrary variables while addressing some of the long-standing data-scaling problems in biopharmaceutical manufacturing or other processes.

### SUMMARY

To address some of the limitations of the current best industrial practices, described herein are embodiments relating to systems and methods that improve upon traditional techniques for data scaling, transfer, and/or amplification of biopharmaceutical or other processes. “Data scaling” generally refers to the process of discovering and/or applying mathematical relationships between two data sets, which may be referred to as a “source” data set and a “target” data set. With data scaling, a linear model uses certain parameters (e.g., slope and intercept) to capture the scaling relationship between the source and target data sets. Scaling models, and the process of developing such models, can provide certain insights and have various use cases.

One such use case is “data transfer,” which generally refers to the process of transferring data from one process (a “source” process) to another (a “target” process). For example, the source and target processes may be biopharmaceutical processes associated with different sites, scales, and/or drug products. As a more specific example, voluminous experimental data from a bench-top scale (e.g., 2 liter) bioreactor may be scaled/transferred to a pilot scale (e.g., 500 liter) or commercial scale (e.g., 20,000 liter) bioreactor, with the latter having very limited experimental data, in order to generate a predictive or inferential model (e.g., a machine learning model such as a regression model or neural network) for the larger-scale target process. In some embodiments, the data transfer process is purposely interfered with in a manner that causes the target data set to have certain desired properties (e.g., to control the variability of the transferred data), in what is generally referred to herein as “data amplification.” This may be done by manually changing certain parameters of the data scaling model to achieve the desired properties.

The data scaling, transfer, and/or amplification process can effectively reuse or repurpose data that is available from source processes, thereby significantly reducing the time required to generate, calibrate, and/or maintain models for target processes, especially in situations such as the development and/or manufacture of pipeline drugs that have little or no past production history. Numerous other use cases are also possible, some of which are described in greater detail below.

In some embodiments, a method for scaling data across different processes includes obtaining first time-series data indicative of one or more input, state, and/or output parameters of a first process over time, and obtaining second time-series data indicative of one or more input, state, and/or output parameters of a second process over time. The method also includes generating, by one or more processors, a scaling model specifying time-varying scaling relationships between the input, state, and/or output parameters of the first process and the input, state, and/or output parameters of the second process. The method also includes transferring, by the one or more processors and using the scaling model, source time-series data associated with a source process to target time-series data associated with a target process. The source time-series data is indicative of one or more input, state, and/or output parameters of the source process over time, and the target time-series data is indicative of one or more input, state, and/or output parameters of the target process over time. The method also includes storing, by the one or more processors, the target time-series data in memory.

In another embodiment, a system includes one or more processors and one or more computer-readable media storing instructions. When executed by the one or more processors, the instructions cause the one or more processors to obtain first time-series data indicative of one or more input, state, and/or output parameters of a first process over time, and obtain second time-series data indicative of one or more input, state, and/or output parameters of a second process over time. The instructions also cause the one or more processors to generate a scaling model specifying time-varying scaling relationships between the input, state, and/or output parameters of the first process and the input, state, and/or output parameters of the second process. The instructions also cause the one or more processors to transfer, using the scaling model, source time-series data associated with a source process to target time-series data associated with a target process. The source time-series data is indicative of one or more input, state, and/or output parameters of the source process over time, and the target time-series data is indicative of one or more input, state, and/or output parameters of the target process over time. The instructions also cause the one or more processors to store the target time-series data in memory.

### BRIEF DESCRIPTION OF THE DRAWINGS

The skilled artisan will understand that the figures described herein are included for purposes of illustration and do not limit the present disclosure. The drawings are not necessarily to scale, and emphasis is instead placed upon illustrating the principles of the present disclosure. It is to be understood that, in some instances, various aspects of the described implementations may be shown exaggerated or enlarged to facilitate an understanding of the described implementations. In the drawings, like reference characters throughout the various drawings generally refer to functionally similar and/or structurally similar components.

FIG. 1 is a simplified block diagram of an example system that can implement one or more of the data scaling, transfer, and/or amplification techniques described herein.

FIG. 2 depicts normalized oxygen flow rate profiles for example bioreactor processes run at two different scales.

FIG. 3 is a flow diagram of an example method for scaling data across different processes.

FIGS. 4A-D depict normalized oxygen flow rate profiles, estimated scaling factors and uncertainties, and actual and estimated (normalized) target signals in a use case where the source process is a 300 liter pilot-scale bioreactor process for biologic production and the target process is a 10,000 liter commercial-scale bioreactor process for biologic production.

FIGS. 5A-C depict normalized viable cell density (VCD) profiles for a biologic produced in a 2,000 liter commercial-scale bioreactor and a 2 liter small-scale bioreactor, with estimated scaling factors.

FIGS. 6A-D depict normalized oxygen flow rate profiles for producing six source products and one target product, similarity measures for each source product relative to the target product, and source product rankings with respect to the target product.

FIGS. 7A-C depict product sieving performance of a hollow membrane fiber in a 50 liter perfusion bioreactor installed with an alternating tangential flow (ATF) system, corresponding (normalized) Raman spectral scans from the bioreactor and permeate, and a similarity measure between the Raman spectral scans as a function of time.

FIG. 8 depicts example scaling relationships between different products and scales.

FIGS. 9A-B depict an actual, normalized oxygen flow rate profile for a source process at a 300 liter scale, and actual versus predicted normalized oxygen flow rate profiles at a 15,000 liter scale.

### DETAILED DESCRIPTION

The various concepts introduced above and discussed in greater detail below may be implemented in any of numerous ways, and the described concepts are not limited to any particular manner of implementation. Examples of implementations are provided for illustrative purposes.

### Example System

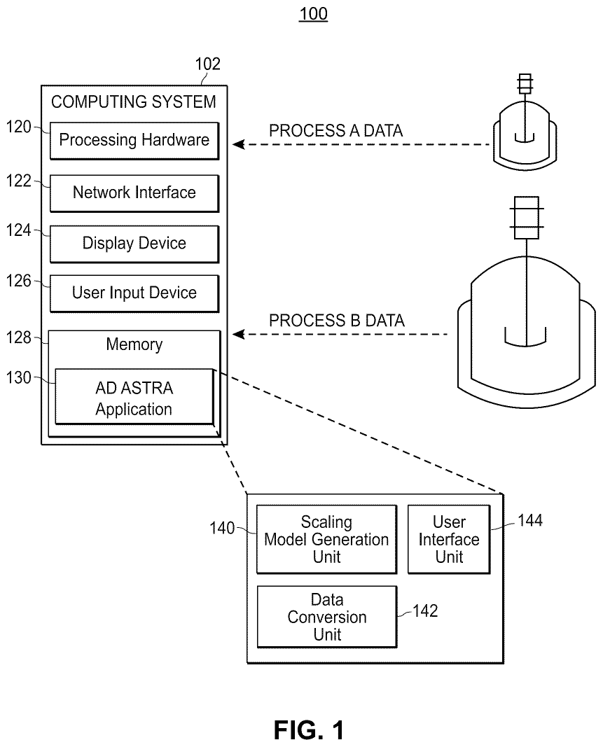

FIG. 1 is a simplified block diagram of an example system 100 that can be used to amplify, scale, and transfer data from a first process (“Process A”) to a second process (“Process B”). As used herein, the term “scale” or “scaling” may be used to refer to the operation of transferring or projecting data from one process to another (e.g., Process A to Process B), or to refer to the relative physical size of equipment and/or materials associated with processes. To clarify which usage is intended, the former meaning is primarily referred to herein in connection with “data,” “parameters,” or “variables” (e.g., “scaled data/parameters/variables” or “scaling data/parameters/variables”), while the latter is primarily referred to herein with reference to a process involving one or more physical objects (e.g., “scaling up” a bioreactor process).

FIG. 1 depicts an example embodiment in which Process A and Process B are bioreactor processes (for producing/growing a biopharmaceutical drug product) that use bioreactors of different sizes, and thus have different amounts of contents. Each bioreactor discussed herein may be any suitable vessel, device, or system that supports a biologically active environment, which may include living organisms and/or substances derived therefrom (e.g., a cell culture) within a media. The bioreactor may contain recombinant proteins that are being expressed by the cell culture, e.g., such as for research purposes, clinical use, commercial sale, or other distribution. Depending on the biopharmaceutical process, the media may include a particular fluid (e.g., a “broth”) and specific nutrients, and may have a target pH level or range, a target temperature or temperature range, and so on. Collectively, the contents and parameters/characteristics of media are referred to herein as the “media profile.”

In “upscaling” scenarios or embodiments, Process A uses a smaller-scale bioreactor and Process B uses a larger-scale bioreactor. For example, Process A may use a 2 liter bench-top scale bioreactor and Process B may use a 500 liter pilot-scale bioreactor, or Process A may use a 500 liter pilot-scale bioreactor and Process B may use a 20,000 liter commercial-scale bioreactor, etc. “Downscaling” scenarios or embodiments are also possible, with Process A using a larger-scale bioreactor than Process B (e.g., for small-scale model qualification, as discussed below).

In other embodiments, Process A and Process B can differ from each other in other (or additional) ways. For example, Process A may be a bioreactor process for producing a particular biopharmaceutical drug product at a first site (e.g., a first manufacturing facility), and Process B may be a bioreactor process for producing the same biopharmaceutical drug product at a different, second site (e.g., a second manufacturing facility). Additionally or alternatively, Process A may be a bioreactor process for producing/growing a first biopharmaceutical drug product, and Process B may be a bioreactor process for producing/growing a different, second biopharmaceutical drug product. In still other embodiments, Process A and Process B may involve the use of equipment other than bioreactors, such as purification or filtration systems of different sizes, for example.

In still other embodiments, Process A and Process B are not biopharmaceutical processes. For example, Process A and Process B may be processes for developing or manufacturing a small-molecule drug product or products, or industrial processes entirely unrelated to pharmaceutical development or production (e.g., oil refining processes with Processes A and B using different operating parameters and/or different types of refining equipment, etc.).

The system 100 includes a computing system 102, which in this example includes processing hardware 120, a network interface 122, a display device 124, a user input device 126, and memory 128. Processing hardware 120 includes one or more processors, each of which may be a programmable microprocessor that executes software instructions stored in the memory 128 to perform some or all of the functions of the computing system 102 as described herein. Alternatively, one or more of the processors in processing hardware 120 may be other types of processors (e.g., application-specific integrated circuits (ASICs), field-programmable gate arrays (FPGAs), etc.). The memory 128 may include one or more physical memory devices or units containing volatile and/or non-volatile memory. Any suitable memory type or types may be used, such as read-only memory (ROM), solid-state drives (SSDs), hard disk drives (HDDs), and so on. In some embodiments, a portion of the memory 128 stores an operating system, another portion of the memory 128 stores instructions of software applications, and another portion of the memory 128 stores data used and/or generated by the software applications (e.g., any of the time-series data or “signals” discussed herein).

The network interface 122 may include any suitable hardware (e.g., front-end transmitter and receiver hardware), firmware, and/or software configured to communicate via one or more networks using suitable communication protocols. For example, the network interface 122 may be or include an Ethernet interface. Generally, the network interface 122 may enable the computing system 102 to receive data relating to Process A (and possibly Process B and/or other processes) from one or more local or remote sources (e.g., via one or more wired and/or wireless local area networks (LANs), and/or one or more wired and/or wireless wide area networks (WANs) such as the Internet or an intranet).

The display device 124 may use any suitable display technology (e.g., LED, OLED, LCD, etc.) to present information to a user, and the user input device 126 may include a keyboard or other suitable input device (e.g., microphone). In some embodiments, the display device 124 and the user input device 126 are integrated within a single device (e.g., a touchscreen display). Generally, the display device 124 and the user input device 126 may combine to enable a user to interact with user interfaces (e.g., a graphical user interface (GUI)) generated by the processing hardware 120.

As noted above, the memory 128 can store the instructions of one or more software applications. One such application is an automatic data amplification, scaling and transfer (AD ASTRA) application 130. The AD ASTRA application 130, when executed by the processing hardware 120, is generally configured to generate scaling models that specify time-varying scaling relationships between process data associated with different processes, such as Process A and Process B, and to project/transfer (and possibly amplify) data across processes using such scaling models. The process data can include time-series data indicative of one or more process input parameters, one or more process state parameters, and/or one or more process output parameters, across a number of time intervals (e.g., one value per day, one value per hour, etc.). The processes from which and to which the AD ASTRA application 130 transfers data are referred to herein as the “source process” and “target process,” respectively, and the data associated with those processes is referred to herein as “source data” (or “source time-series data,” etc.) and “target data” (or “target time-series data,” etc.), respectively. Thus, in the example of FIG. 1, Process A is the source process and Process B is the target process.

The AD ASTRA application 130 includes a scaling model generation unit 140 configured to generate a scaling model based on at least one set of experimental data from each of Process A and Process B. The AD ASTRA application 130 also includes a data conversion unit 142 configured to transfer/scale data from Process A to Process B using the generated scaling model. In some embodiments, the AD ASTRA application 130 is flexible enough to generate scaling models, and transfer/scale data, for a wide variety of source/target processes and/or use cases. The AD ASTRA application 130 also includes a user interface unit 144 configured to generate a user interface (which can be presented on the display device 124) that enables a user to interact with the scaling/conversion process. For example, the user interface may enable a user to manually select source and/or target processes/datasets, set parameters that change the variance of (i.e., amplify) the source data, and/or view source and/or target data (and/or metrics associated with that data).

The parameters operated upon by the AD ASTRA application 130 depend upon the nature of Process A and Process B, and the use case. For example, one general use case is to develop a machine learning model that predicts or infers product quality attributes or other parameters of Process B (e.g., yield, titer, future glucose or other metabolite concentration(s), etc.) based on measurable media profile and/or other parameters of Process B (e.g., pH, temperature, current metabolite concentration(s), etc.), in order to control certain inputs to Process B (e.g., glucose feed rate) or for other purposes (e.g., to assist in the design of Process B). If few experiments have been run for Process B, it may be difficult or impossible to create a reliable predictive or inferential model of that sort using only the experimental data from Process B. Thus, the scaling model generation unit 140 may generate a scaling model that transfers the Process A data reflecting parameters to be used as inputs to the predictive or inferential model (e.g., pH, temperature, current metabolite concentration(s), etc.) into analogous data for Process B. Various example use cases are discussed in more detail below.

It is understood that other configurations and/or components may be used instead of (or in addition to) those shown in the system 100 of FIG. 1. For example, a first other computing system may transmit Process A and Process B data to the computing system 102, and/or a second other computing system may receive scaled/transferred data from the computing system 102, and possibly use (or facilitate the use of) the scaled data (e.g., to train and/or use a machine learning model such as the predictive or inferential model noted above, or any other suitable application). Alternatively, computing system 102 itself may include these other (possibly distributed) computing devices. The system 100 may also include instrumentation for measuring parameters in Process A and/or Process B (e.g., Raman spectroscopy systems with probes, flow rate sensors, etc.), and/or for controlling parameters in Process A and/or Process B (e.g., glucose pumps, devices with heating and/or cooling elements, etc.).

In some embodiments, the AD ASTRA application 130 can compare any two parameters given their time-series data. The techniques applied by the AD ASTRA application 130 may be purely data-based, without requiring any prior knowledge of how parameters are related, or whether the parameters are related at all. This may provide flexibility in addressing certain long-standing data-scaling problems in biopharmaceutical manufacturing or other processes, some examples of which are discussed below.

### Example Scaling Model

The scaling model generated by the scaling model generation unit 140 of FIG. 1 will now be discussed in more detail, according to various embodiments. To address the issues with similitude-based scaling models (discussed above in the Background section), the scaling model generation unit 140 applies an improved data-based framework to calculate optimal (in some embodiments) scaling between any arbitrary variables.

First, let {Xₜ}_{t∈N} and {Yₜ}_{t∈N} denote two generic signals, which are assumed to be related to the following model:

$${Y}_{t} = α + β {X}_{t} + {e}_{t}^{y} \qquad ( Equation 2 a )$$

where: Cₜ≡[1, Xₜ]∈ [symbol] ; θ≡[α, β]^{T}∈θ⊆ [symbol] is a vector of scaling parameters; and

$${e}_{𝔱}^{y} ∼ \text{?} ( 0 , {σ}_{t}^{2} )$$

is a sequence of independent Gaussian noise with zero mean and variance,

$${σ}_{𝔱}^{2} ∈ ℝ .$$

Physically, α∈ [symbol] denotes the bias and β∈ [symbol] denotes the slope between the two signals.

The model in Equation 2a is referred to herein as a scaling model, because it establishes the scaling relationship between the signals, where [symbol] and [symbol] are the “target” and “source” signals, respectively. Here, it is assumed that the target and source are arbitrary signals (though in practice their selection is guided by the use case, as discussed in further detail below), and one-dimensional. θ completely defines the scaling relationship between the two signals. In practice, θ is often unknown and needs to be estimated. Now, given Equation 2a and the data sequences (or time-series data) [symbol] and [symbol] , the objective is to estimate θ. For simplicity, let [symbol] ={x, y}, where x=[x₁, x₂, . . . , x_{T}]^{T}, where y=[y₁, y₂ . . . , y_{T}]^{T}, and T is the length of the signal. Given [symbol] , the optimal solution to the parameter estimation problem in Equation 2a is provided by the ordinary least-squares (OLS) method or the maximum-likelihood (ML) method. See, e.g., Montgomery et al., 2012, Introduction to Linear Regression Analysis, John Wiley & Sons, vol. 821. For example, rearranging Equation 2a using a vector notation, one can write

$$y = c θ + {e}^{y} , \qquad ( Equation 3 )$$

where c=[1_{Tx1}, x]∈ [symbol] and

$${e}^{y} = {[ {e}_{1}^{y} , {e}_{2}^{y} , … , {e}_{T}^{y} ]}^{T}$$

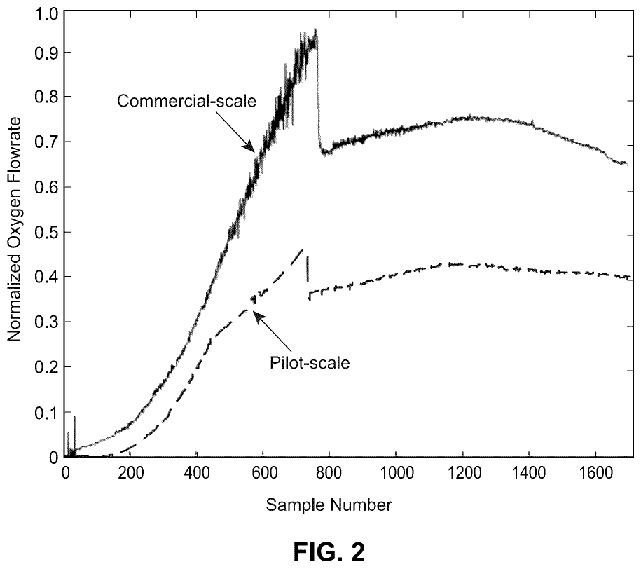

∈ [symbol] . The OLS estimation of θ in Equation 3 is given as follows

$$\hat{θ} = {( {c}^{T} c )}^{- 1} {c}^{T} y , \qquad ( Equation 4 )$$

where \hat{θ} is an OLS estimate of θ. Note that while Equation 4 gives an analytical approach to compute the scaling parameters, the scaling model of Equation 2a has a limited scope of application. This is because the scaling model of Equation 2a assumes a uniform scaling between x and y, such that θ remains constant for all t=1, 2, . . . , T. In reality, non-uniform (time-varying) scaling is a common occurrence in biopharmaceutical manufacturing. For example, the oxygen demand for a biotherapeutic protein produced at a pilot scale and at a commercial bioreactor scale is different due to different operating conditions. The oxygen demand in the bioreactors is comparable at the start of the campaign, but as the cells start to grow the demand in the commercial bioreactor outpaces that in the pilot bioreactor. FIG. 2 shows representative, normalized oxygen flow rates in commercial-scale and pilot-scale bioreactors, corresponding to target and source signals (parameter values), respectively. While normalized values are depicted in figures of this disclosure, it is understood that scaling parameters may be generated using process data that is not normalized (or using normalized process data, so long as both the source and target process data are normalized using the same normalizing factor). It is evident from FIG. 2 that the scaling between the signals/values is non-uniform over time. To allow for non-uniform scaling between the target and source signals, Equation 2a is refined as follows:

$$\begin{matrix}{Y}_{t} = {α}_{t} + {β}_{t} {X}_{t} + {e}_{t}^{y} & ( Equation 5 a ) \\ = {C}_{t} {θ}_{t} + {e}_{t}^{y} , & ( Equation 5 b )\end{matrix}$$

where: θₜ=[αₜ, βₜ]^{T}∈θ⊆ [symbol] is a vector of time-varying scaling factors. The scaling parameters in Equation 5b capture the time-varying scaling relationship between the target and source signals. A standard approach for parameter estimation in models having the general form of Equation 5b is to formulate the estimation problem as an adaptive learning problem. Adaptive methods, such as block-wise linear least-squares or moving/sliding window least squares (MWLS) (Kadlec et al., 2011, Computers & Chemical Engineering, at 35:1-24), recursive least-squares (RLS) (Jiang and Zhang, 2004, Computers & Electrical Engineering, 30:403-416), recursive partial least-squares (RPLS) (Dayal et al., 1997, Journal of Chemometrics, 11:73-85), locally weighted least squares (LWLS) (Ge and Song, 2010, Chemometrics and Intelligent Laboratory Systems, 104:306-317), and smoothed passive-aggressive algorithm (SPAA) (Sharma et al., 2016, Journal of Chemometrics, 30:308-323) have been proposed for such learning. While the scaling model generation unit 140 may use any of these techniques, in some embodiments, these techniques are recursive methods that are efficient in estimating constant (or “slowly” varying) parameters recursively in time, as opposed to time-varying parameters. Furthermore, with existing methods, it is non-trivial to include a priori information available on the parameters. To address these issues, the scaling model generation unit 140 may instead use a Bayesian framework for parameter estimation in Equation 5b.

Unlike frequentist methods (e.g., OLS or ML) that assume {θₜ}_{t∈N} as deterministic, under a Bayesian formulation, {θₜ}_{t∈N} is considered a random variable with some initial density θ₀~p(θ₀). The initial density captures the a priori information available on the parameters. For example, if the scaling parameters are assumed to lie within some interval-constraints, then a uniform or Gaussian density can be defined over the given intervals.

Given p(θ₀) and [symbol] , a Bayesian approach seeks to compute a posterior density for {θₜ}_{t∈N}. A posterior density can be constructed both under real-time (or “online”) and non-real-time (or “offline”) settings. To distinguish between the two settings, one can define [symbol] ={x_{1:t}, y_{1:t}}, where x_{1:t}=[x₁, x₂, . . . , xₜ]^{T} and y_{1:t}=[y₁, y₂, . . . yₜ]^{T}. Now, for real-time estimation in Equation 5b, a filtering posterior density {p(θ| [symbol] )}_{t∈N} is recursively computed. The filtering density encapsulates all the information about the unknown parameter θₜ given [symbol] . To compute p(θₜ| [symbol] ), information only up until time t is used. The filtering formulation is particularly useful in applications where real-time scaling relationships are required. For offline estimation, a Bayesian method seeks to compute a smoothing posterior density {p(θₜ| [symbol] )}_{t∈N}. Again, to compute p(θₜ| [symbol] ), all information up until time Tis used. For ease of explanation, real-time learning is addressed here. It is understood, however, that similar techniques and/or calculations may be used for offline learning.

To calculate the filtering density for the parameters, Equation 5b is represented using a stochastic state-space model (SSM) formulation, as given below:

$${θ}_{t + 1} = {A}_{t} {θ}_{t} + {e}_{t}^{θ} , \qquad ( Equation 6 a )$$

$${Y}_{t} = {C}_{t} {θ}_{t} + {e}_{t}^{y} , \qquad ( Equation 6 b )$$

where

$${\{ {e}_{t}^{θ} \}}_{t ∈ N} and {\{ {e}_{t}^{y} \}}_{t ∈ N}$$

are mutually independent sequences of independent random variables, such that

$${e}_{t}^{θ} ∼ 𝒩 ( \cdot \vert 0 , {Q}_{t} )$$

is a multivariate Gaussian noise with zero mean and covariance Qₜ∈

$${𝕊}_{+}^{2 x 2} and {e}_{t}^{y} ∼ 𝒩 ( \cdot \vert 0 , {R}_{t} )$$

is a Gaussian noise with zero mean and variance Rₜ= [symbol] . Further Aₜ∈ [symbol] and Cₜ∈ [symbol] are system matrices. In contrast to the scaling model in Equation 5b, the SSM representation in Equations 6a and 6b assumes an artificial dynamics model for the scaling parameters (see Equation 6a). Under the Bayesian paradigm, introducing artificial dynamics is important for adequate exploration of the parameter space. See Tulsyan et al., 2013, Journal of Process Control, 23:516-526. The dynamics of the scaling parameters in Equation 6a are completely defined by Aₜ and

$${e}_{t}^{θ} .$$

For a Gaussian noise,

$${e}_{t}^{θ} ,$$

and Aₜ=I_{2×2} for all t∈N, Equation 6a represents a random-walk model.

In the SSM formulation of the scaling model in Equations 6a and 6b, {θₜ}_{t∈N} represents the states, {Yₜ}_{t∈N} is the measurement, and {Xₜ}_{t∈N} is the parameter. In Equations 6a and 6b, {θₜ}_{t∈N} and {Yₜ}_{t∈N} are θ(⊆ [symbol] ) and y(⊆ [symbol] ) valued stochastic processes, respectively, defined on a probability space (Ω, [symbol] , [symbol] ). The discrete-time state process {θₜ}_{t∈N} is an unobserved Markov process, with initial density θ₀~p(θ₀) and Markovian transition density p(θ′|θ), such that

$${θ}_{0} ∼ p ( {θ}_{0} ) \qquad ( Equation 7 a )$$

$${θ}_{t + 1} \vert {θ}_{t} ∼ p ( {θ}_{t + 1} \vert {θ}_{t} {A}_{t} ) \qquad ( Equation 7 b )$$

for all t∈ [symbol] . The state process {θₜ}_{t∈N} is hidden but observed through {Yₜ}_{t∈N}. Further, {Yₜ}_{t∈N} is conditionally independent given {θₜ}_{t∈N} with marginal density p(y|θ. x), such that

$${Y}_{t} \vert ( {θ}_{0} , … {θ}_{t} ) ∼ p ( {y}_{t} \vert {θ}_{t} , {C}_{t} ) \qquad ( Equation 8 )$$

for all t∈ [symbol] . All the density functions in Equations 7a, 7b, and 8 are with respect to a suitable dominating measure, such as a Lebesgue measure. Given the scaling model in Equations 6a and 6b, the measurement sequence {Yₜ}_{t∈N}, and the parameter sequence {Xₜ}_{t∈N}, the objective is to estimate the states {θₜ}_{t∈N}. As discussed earlier, under the Bayesian framework, this entails recursively computing the filtering density {p(θₜ|y_{1:t})}_{t∈N}. Now, using the Bayes' rule, p(θₜ|y_{1:t}) can be written as

$$\begin{matrix}p ( {θ}_{t} \vert {y}_{1 : t} ) = \frac{p ( {y}_{t} \vert {θ}_{t} ) p ( {θ}_{t} \vert {y}_{1 : t - 1} )}{p ( {y}_{t} \vert {y}_{1 : t - 1} )} , & ( Equation 9 a ) \\ ∝ p ( {y}_{t} \vert {θ}_{t} ) p ( {θ}_{t} \vert {y}_{1 : t - 1} ) , & ( Equation 9 b )\end{matrix}$$

where p(yₜ|θₜ) is the likelihood function, p(θₜ|y_{1:t-1}) is the predicted posterior density, and p(yₜ|y_{1:t-1}) is a normalizing constant. Using the law of marginalization, the predicted posterior density can be calculated as

$$\begin{matrix}p ( {θ}_{t} \vert {y}_{1 : t - 1} ) = {\int }_{θ} p ( {θ}_{t} , {θ}_{t - 1} \vert {y}_{1 : t - 1} ) d {θ}_{t - 1} , & ( Equation 10 a ) \\ = {\int }_{θ} p ( {θ}_{t} \vert {θ}_{t - 1} ) p ( {θ}_{t - 1} \vert {y}_{1 : t - 1} ) d {θ}_{t - 1} , & ( Equation 10 b )\end{matrix}$$

where p(θₜ|θₜ₋₁) is the transition density and p(θₜ₋₁|y_{1:t-1}) is a filtering density at t−1. Equations 9b and 10b give a recursive approach to calculate {p(θₜ|Y_{1:t})}_{t∈N}. To compute a point estimate from {p(θₜ|y_{1:t})}_{t∈N}, a common approach is to minimize the mean-square error (MSE) risk function [symbol] ≡ [symbol] _{(θ}_{ₜ}_{|y}_{_{1:t}}∥θₜ−θ_{t|t}∥², where θ_{t|t}∈ [symbol] is a point estimate of θₜ∈θ. It can be shown that minimizing [symbol] yields [symbol] [θₜ|y_{1:t}], the posterior mean as the optimal estimate, such that

$${θ}_{t \vert t} ≡ 𝔼 [ {θ}_{t} \vert {y}_{1 : t} ] = {\int }_{θ} {θ}_{t} p ( {θ}_{t} \vert {y}_{1 : t} ) d {θ}_{t} , \qquad ( Equation 11 )$$

where θ_{t|t}∈ [symbol] is the posterior mean. See Tulsyan et al., 2013, J. Process Control, 23:516-526. For the posterior mean in Equation 11, it is possible to compute the posterior variance as

$${P}_{t \vert t} ≡ 𝕍 [ {θ}_{t} \vert {y}_{1 : t} ] = {\int }_{θ} {[ {θ}_{t} - {θ}_{t \vert t} ] [ {θ}_{t} - {θ}_{t \vert t} ]}^{T} p ( {θ}_{t} \vert {y}_{1 : t} ) d {θ}_{t} , \qquad ( Equation 12 )$$

where P_{t|t}∈ [symbol] is the posterior variance. The posterior variance in Equation 12 is commonly selected as a measure to quantify the quality of the point estimate in Equation 11, with smaller posterior variance corresponding to higher confidence in the point estimate. Calculating the estimates in Equations 11 and 12 requires recursive evaluation of Equations 9b and 10b. Fortunately, for the linear SSM in Equations 9a and 9b, and for the choice of a Gaussian prior, p(θ₀), the densities in Equations 9b and 10b can be analytically solved using the Kalman filter. See Kalman et al., 1960, Journal of Basic Engineering, 82:35-45. It can be shown that for a linear Gaussian SSM, the densities in Equations 9b and 10b are Gaussian, such that

$$p ( {θ}_{t - 1} \vert {y}_{1 : t - 1} ) = \text{?} ( {θ}_{t - 1} \vert {θ}_{t - 1 \vert t - 1} , {P}_{t - 1 \vert t - 1} ) , \qquad ( Equation 13 a )$$

$$p ( {θ}_{t} \vert {y}_{1 : t} ) = \text{?} ( {θ}_{t} \vert {θ}_{t} - {θ}_{t \vert t - 1} , {P}_{t \vert t - 1} ) , \qquad ( Equation 13 b )$$

$$p ( {θ}_{t} \vert {y}_{1 : t} ) = \text{?} ( {θ}_{t} \vert {θ}_{t \vert tt} , {P}_{t \vert t} ) , \qquad ( Equation 13 c )$$

### See Chen et al., Statistics, 2003, at 182:1-69.

The Kalman filter propagates the mean and covariance functions (the sufficient statistics for Gaussian distributions) through the update (Equation 13a) and prediction (Equation 13b) steps to calculate the posterior density in Equation 13c. This is outlined below in Algorithm 1. The Kalman filter yields a minimum mean-square error for the state estimation problem in Equations 6a and 6b. In other words, Algorithm 1 is optimal in MSE for all t∈N. See Chen et al., 2003, Statistics, 182:1-69. Moreover, conditioning (Equation 11) on past measurements reduces the effect of noisy measurements and parameters on state estimates.

Algorithm 1, which may be implemented by the AD ASTRA application 130 in some embodiments, is as follows:

Algorithm 1: Kalman Filter

| | |
|---|---|
| 1. | Input: Scaling model: Aₜ, Cₜ, Yₜ, Rₜ, Qₜ, θ_{0\|0}, P_{0\|0} |
| 2. | Output: State estimates θ_{t\|t} |
| 3. | Initialize: θ₀~ [symbol] (· \|θ_{0\|0}, P_{0\|0}) |
| 4. | for t = 1 to T do |
| 5. | Predicted mean: θ_{t\|t-1} = Aₜθ_{t-1\|t-1} |
| 6. | $Predicted covariance : {P}_{t \vert t - 1} = {Q}_{t - 1} + {A}_{t} {θ}_{t - 1 \vert t - 1} {A}_{t}^{T}$ |
| 7. | $Innovation : {S}_{t} = {C}_{t} {P}_{t \vert t - 1} {C}_{t}^{T} + {R}_{t}$ |
| 8. | $Kalman gain : {K}_{t} = {P}_{t \vert t - 1} {C}_{t}^{T} {S}_{t}^{- 1}$ |
| 9. | Updated mean: θ_{t\|t-1} = θ_{t-1\|t-1} + Kₜ(yₜ − Cₜθ_{t-1\|t-1}) |
| 10. | Updated covariance: P_{t\|t} = P_{t\|t-1} − KₜCₜP_{t\|t-1} |
| 11. | end for |

In Algorithm 1, Aₜ, Qₜ, θ_{0|0} and P_{0|0} are user-defined parameters that allow for a variety of a priori knowledge to be included. For example, if we know a priori that the slope between the target and source signals is time-varying, but has a fixed bias, i.e., θ_{1,t}=a, where θₜ=[θ_{1,t}, θ_{2,t}]^{T} for all t∈N, then this information can be included in Equations 6a and 6b by defining

$${θ}_{0 \vert 0} = [ \begin{matrix}a \\ {b}_{1}\end{matrix} ] , {P}_{0 \vert 0} = [ \begin{matrix}0 & 0 \\ 0 & {b}_{2}\end{matrix} ] , {A}_{t} = [ \begin{matrix}0 & 0 \\ 0 & {b}_{3}\end{matrix} ] , {Q}_{t} = [ \begin{matrix}0 & 0 \\ 0 & {b}_{4}\end{matrix} ] , \qquad ( Equation 14 )$$

where b₁, b₂, b₃, b₄∈ [symbol] are known constants. Using Algorithm 1 with the definition of Equation 14 ensures that θ_{1,t}=a for all t∈ [symbol] , while θ_{2,t} is optimally estimated using the Kalman filter. The scaling model in Equations 6a and 6b is flexible enough to include a variety of complex a priori information. While Algorithm 1 is for online (real-time) estimation of optimal scaling between target and source signals, it is to be understood that offline estimation is also possible in certain embodiments and/or scenarios.

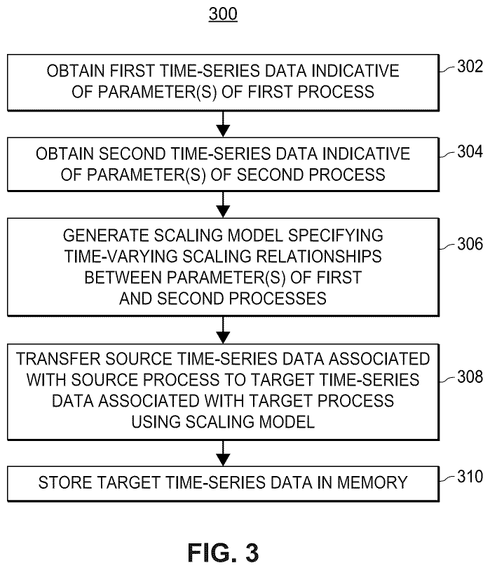

FIG. 3 is a flow diagram of an example method 300 for scaling data across different processes. The method 300 may be performed in whole or in part by the computing system 102 of FIG. 1 (e.g., by the processing hardware 120 when executing instructions of the AD ASTRA application 130 stored in the memory 128), for example.

At block 302, first time-series data indicative of one or more parameters of a first process is obtained. The first time-series data is indicative of one or more input parameters (e.g., feed rate), state parameters (e.g., metabolite concentration), and/or output parameters (e.g., yield) of the first process. Block 302 may include retrieving the first time-series data from a database in response to a user selecting a particular data set via the user input device 126, display device 124, and user interface unit 144, for example. The parameter(s) represented by the first time-series data may be the parameters of any of the “source” data sets discussed above with reference to various use cases, for example.

At block 304, second time-series data indicative of one or more parameters of a second process is obtained. The second time-series data is indicative of one or more input, state, and/or output parameters of the second process (e.g., the same type(s) of parameters as are obtained at block 302 for the first process). Block 304 may include retrieving the second time-series data from a database in response to a user selecting a particular data set via the user input device 126, display device 124, and user interface unit 144, for example. The parameter(s) represented by the second time-series data may be the parameters of any of the “target” data sets discussed above with reference to various use cases, for example.

At block 306, a scaling model that specifies time-varying scaling relationships between the parameter(s) of the first and second processes is generated. The scaling model may be any of the models (with time-varying scaling) disclosed herein, for any of the use cases discussed above, for example, or may be another suitable scaling model built upon similar principles. Preferably, the scaling model is a probabilistic estimator, such as the Kalman filter discussed above (or an extended Kalman filter, etc.).

At block 308, using the scaling model generated at block 306, source time-series data associated with a source process is transferred to target time-series data associated with a target process. The source time-series data is indicative of one or more input, state, and/or output parameters of the source process over time, and the target time-series data is indicative of input, state, and/or output parameters of the target process over time. Block 308, in part or in its entirety, may occur substantially in real-time as the source time-series data is obtained, or as a batch process, etc.

At block 310, the target time-series data is stored in memory (e.g., in a different unit, device, and/or portion the memory 128). For example, the target time-series data may be stored in a local or remote training database, for use (e.g., in an additional block of the method 300) to train a machine learning (predictive or inferential) model for use with the target process (e.g., for monitoring, such as monitoring of metabolite concentrations or product sieving, and/or for control, such as glucose feed rate control).

As non-limiting examples, the parameters indicated by the first, second, source, and/or target time-series data may include oxygen flow rate, pH, agitation, and/or dissolved oxygen. However, virtually any parameters are possible. In some embodiments and/or use cases, the parameters of the first/source time-series data differ at least in part from the parameters of the second/target time-series data, such that some source parameters are used to determine different target parameters.

In some embodiments and/or use cases, the source time-series data and the source process are the first time-series data and the first process, respectively, and/or the target time-series data and the target process are the second time-series data and the second process, respectively. In other embodiments and/or use cases, however, this is not the case. For example, the scaling model generated at block 306 may relate Process A to Process B, whereas block 308 projects/transfers a different Process C to a different Process D, so long as Process A is sufficiently similar to Process C and Process B is sufficiently similar to Process D (or more precisely, so long as the relation between Process A and Process B is known or expected to be similar to the relation between Process C and Process D). As just one example, Process A may be for a particular drug product, site, and scale, while Process C may be for the same drug product and scale, but at a different site. While this may make the data scaling less accurate in some cases, it may nonetheless be acceptable so long as the different sites are sufficiently similar, or so long as the processes are not overly sensitive to the process site.

In some embodiments and use cases, the first process and source process (which may be the same or different from each other) are associated with a first process site, while the second process and target process (which may be the same or different from each other) are associated with a second, different process site. For example, the first/source process site may be in one manufacturing facility, and the second/target process site may be in another manufacturing facility. Additionally or alternatively, the first process and source process may be associated with a first process scale (e.g., a smaller bioreactor size), and the second process and target process may be associated with a second, different process scale (e.g., a larger bioreactor size). Additionally or alternatively, the first process and source process may be bioreactor processes in which a first biopharmaceutical product grows, and the second process and target process may be bioreactor processes in which a second, different biopharmaceutical product grows.

In some embodiments, the method 300 includes one or more other additional blocks not shown in FIG. 3. For example, the method 300 may include an additional block in which a machine learning model of the target process is generated using the target time-series data (e.g., a predictive or inferential neural network or regression model, etc.), and possibly another block in which one or more inputs to the target process (e.g., a feed rate, etc.) are controlled using the trained machine learning model.

As another example, the method 300 may include, at some point before block 308 occurs, a first additional block in which additional time-series data (indicative of one or more input, state, and/or output parameters of one or more additional processes over time) is obtained, a second additional block in which one or more additional scaling models (each specifying a time-varying relationship between the input, state, and/or output parameters of the first process and the input, state, and/or output parameters of a respective one of the one or more additional processes) is/are generated, and a third additional block in which, based on the scaling model from block 306 and the additional scaling model(s), it is determined that the parameter(s) of the second process have the closest measure of similarity to the input, state, and/or output parameters of the first process (i.e., closer than the additional process(es)). The determination may be made using a Kullback-Leibler divergence (KLD) measure of similarity or Weitzman's measure of similarity as discussed above, for example.

As yet another example, the method 300 may include a first additional block in which a user interface is provided to a user (e.g., by the user interface unit 144, via the display device 124), and a second additional block in which a control setting is received from the user via the user interface. In such an embodiment, block 306 may include using the control setting to set a covariance when generating the scaling model.

It is understood that the blocks shown in FIG. 3 need not occur in the order shown. For example, block 304 may be before or concurrent with block 302, and/or block 306 may occur in real-time as data is received at blocks 302 and 304, etc.

### Example Use Cases

While Algorithm 1 generally gives an optimal approach to extract scaling information between target and source signals from their corresponding time-series data, the details of the approach are specific to the use case. In this section, several problems in industrial biopharmaceutical manufacturing are presented, each of which can be formulated as a data-scaling problem. The efficacy of Algorithm 1 is then demonstrated on these reformulated problems. The applications/use cases discussed here, which are non-limiting, can be broadly classified into one of the following classes of problems: (1) comparing two signals; (2) comparing multiple signals; (3) predicting missing signals; and (4) generating new signals. Each of these classes present a unique data-scaling challenge and requires appropriate modification of Algorithm 1.

### Use Case 1: Comparing Two Signals

The problem of comparing two parameters/variables (also referred to as “signals”) from their time-series data is one general use case. This is an important class of data-scaling problem that has many practical applications in industrial biopharmaceutical manufacturing and other fields. In general, there are different ways to compare two variables. Here, however, the signals are compared based on how they scale against each other using Algorithm 1. The efficacy of Algorithm 1 is demonstrated below through specific, example use cases, which may be implemented, for example, by the system 100 of FIG. 1.

### Use Case 1, Example A: Process Scale-Up Study

A typical lifecycle of commercial biologic manufacturing involves three different scales of cell-culture operations: bench-top scale, pilot scale, and commercial scale. The cell-culture process is initially developed in bench-top bioreactors, and then scaled up to pilot-scale bioreactors, where the process design and parameters are further refined, and where control strategies are refined/optimized. Finally, the cell-culture process is scaled up to industrial-scale bioreactors for commercial production. See Heath and Kiss, 2007, Biotechnology Progress, 23:46-51. At each stage of process scale-up (from bench-top to pilot-scale and from pilot-scale to commercial scale), the at-scale process performance of the bioreactor is continuously validated against the smaller-scale bioreactor. This is to ensure that the at-scale and smaller-scale cell culture exhibit equivalent productivity and equivalent product quality attributes (PQAs). A successful scale-up operation typically results in profiles for titer concentrations, viable cell density (VCD), metabolite profiles, and glycosylation isoforms that are equivalent for the at-scale and smaller-scale bioreactors. This is primarily achieved by manipulating common process variables, such as oxygen flow rates, pH, agitation, and dissolved oxygen. Studying how these manipulated parameters/variables compare across process scales is critical for assessing at-scale equipment fitness, and aids in devising optimal at-scale control recipes. See Junker, 2004, Journal of Bioscience and Bio-engineering, 97:347-364; Xing et al., 2009, Biotechnology and Bioengineering, 103:733-746.

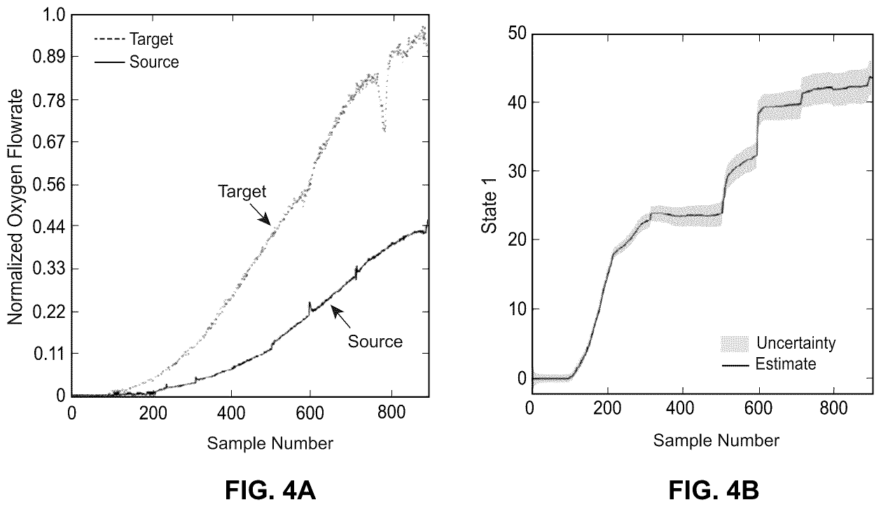

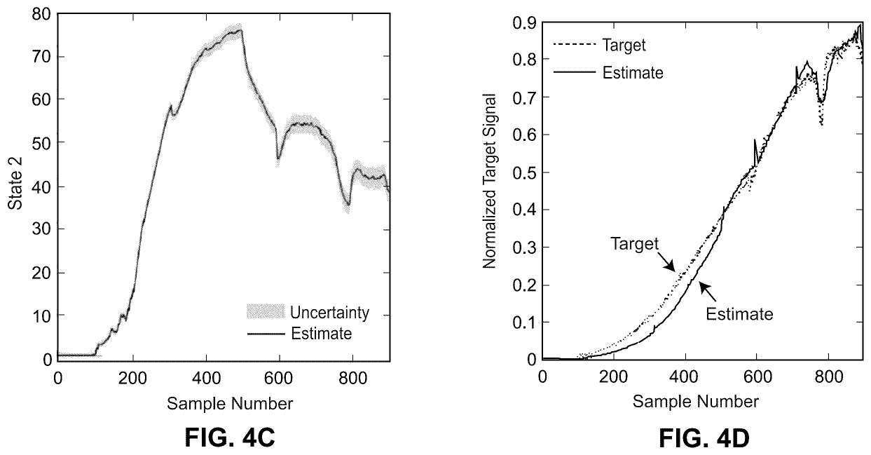

For illustration purposes, the oxygen flow rate profiles for a biologic produced in pilot- and commercial-scale bioreactors are compared. Formally, {Xₜ}_{t∈N} and {Yₜ}_{t∈N} represent the oxygen flow rate profiles for a biologic manufactured in a pilot-scale and a commercial-scale bioreactor, respectively. FIGS. 4A-D depict experimental results for one example implementation in which automatic data amplification, scaling, and transfer techniques disclosed herein were used to estimate target signals for a 10,000 liter commercial-scale bioreactor based on the oxygen flow rate profile for a biologic produced in a 300 liter pilot-scale bioreactor. In the plot of FIG. 4A, the “source” signal represents the measured oxygen flow rate (normalized) for the 300 liter pilot-scale bioreactor, while the “target” signal represents the measured oxygen flow rate (normalized) for the 10,000 liter commercial-scale bioreactor. In biologics manufacturing, oxygen flow rate is a critical manipulated variable for controlling the concentration of dissolved oxygen in the cell-culture. As seen in FIG. 4A, the oxygen flow rate through the commercial-scale (target) bioreactor is higher than in the pilot-scale (source) bioreactor. This is primarily due to the larger volume and higher viable cell count in the commercial-scale bioreactor. The oxygen flow rate is a critical parameter that needs to be continuously monitored as the process is scaled. However, due to the lack of appropriate mathematical tools to continuously monitor scale-up processes, it has traditionally been monitored only at a discrete time using a visual-based analysis. For example, the peak oxygen value (i.e., where the oxygen flow rate is maximum, such as the peak in FIG. 4A), which is also a critical parameter, is compared at different scales to assess the mass transfer efficiency. Despite the complete time-series data being available in FIG. 4A, not much comparative analysis is typically performed except for this peak value analysis.

To address the limitations with existing methods, the AD ASTRA application 130 can use Algorithm 1 to compare {Xₜ}_{t∈N} and {Yₜ}_{t∈N} continuously, and in real-time. First, it is assumed that {Xₜ}_{t∈N} and {Yₜ}_{t∈N} are related according to the SSM of Equations 6a and 6b, with

$${A}_{t} = [ \begin{matrix}1 & 0 \\ 0 & 1\end{matrix} ] , \qquad ( Equation 15 a )$$

$${Q}_{t} = {10}^{- 4} [ \begin{matrix}1 & 0 \\ 0 & 1\end{matrix} ] , \qquad ( Equation 15 b )$$

$${R}_{t} = {10}^{- 4} , \qquad ( Equation 15 c )$$

for all t∈ [symbol] . Equations 15a-15c describe a double random walk model for the process states θₜ=[θ_{1,t}, θ_{2,t}]^{T} in Equation 6a. A single state model, with either pure bias or pure slope can also be obtained by appropriately modifying Aₜ and Qₜ. Now, given Equations 15a-15c, the scaling model between {Xₜ}_{t∈N} and {Yₜ}_{t∈N} is fixed. Next, the scaling model generation unit 140 uses Algorithm 1 to estimate the states θₜ for all t∈ [symbol] , with initial density, θ₀~ [symbol] (θ_{0|0}, P_{0|0}), defined as follows:

$${θ}_{0 \vert 0} = [ \begin{matrix}0 \\ 0\end{matrix} ] , \qquad ( Equation 16 a )$$

$${P}_{0 \vert 0} = [ \begin{matrix}0.5 & 0 \\ 0 & 0.5\end{matrix} ] \qquad ( Equation 16 b )$$

FIGS. 4B and 4C give an estimate of the states, [symbol] and [symbol] , respectively, as calculated by the scaling model generation unit 140 using Algorithm 1. FIGS. 4B and 4C represent scaling factors (the solid lines) with uncertainties (the shaded areas surrounding the solid lines) as calculated using Algorithm 1. It can be seen that the scaling factors are available at each sampling time, as opposed to specific time points as calculated by traditional methods. Moreover, the state estimates are not constant values, but instead time-varying values that represent non-uniform scaling between the signals. FIGS. 4B and 4C show that the bias and the slope between the signals monotonically increase until about sample time t=500, after which the slope starts to decrease (FIG. 4C) but the bias continues to increase (FIG. 4B). Physically, the profiles are much less similar in the first half of the operation than in the second half, where the pilot-scale and commercial-scale bioreactors transition to their respective steady-state operations (separated by a time-varying offset). In FIGS. 4B and 4C, the reliability of the state estimates is established by the small posterior variances. Finally, the estimates obtained with Algorithm 1 are guaranteed to be optimal (in terms of MSE).

Another approach to evaluate the quality of the estimates obtained with Algorithm 1 is to compare the true (actual) and predicted target signals. The predicted target signal, {Y_{t|t}}_{t∈N}, is calculated as Y_{t|t}=Cₜθ_{t|t} for all t∈ [symbol] . FIG. 4D compares the actual and predicted target signals. In FIG. 4D, the “target” trace represents the actual measured oxygen flow rate (normalized) of a 10,000 liter commercial-scale bioreactor, while the “estimate” trace represents the predicted measurements of oxygen flow rate (normalized) using the scaling factors produced by Algorithm 1. As seen in FIG. 4D, the predictions made using the AD ASTRA application 130 in this embodiment were generally in close agreement with the analytical measurements, with a slight offset between the signals in the range (roughly) of sample number 200 to sample number 500. It is possible to achieve an arbitrary level of accuracy in the target signal prediction, however, by tuning model dynamics. Recall that the random-walk model described in Equations 15a-15c, the rate of space exploration by the state process, [symbol] , is controlled by the diagonal elements of Qₜ. By simply increasing Qₜ, the rate of exploration can be made arbitrarily aggressive, thereby yielding improved predictions. From a practical standpoint, increasing Qₜ also leads to noisier scaling factors with higher posterior variances. In other words, while tuning Qₜ allows for improved exploration, caution should be exercised to avoid overfitting. In some embodiments, the user interface unit 144 presents a user interface with a control (e.g., field) that enables a user to set the covariance Qₜ as a control setting (or enables the user to enter some other control setting, such as a position of a slide control, which the AD ASTRA application 130 then uses to derive the covariance Qₜ).

FIGS. 4B-D are unique to the scaling model defined in Equations 15a-15c. Changing the system parameters in Equations 15a-15c and/or 16a-b defines a new model and yields different state estimates. Since the scaling is model dependent, ascribing any meaningful physical interpretations to the results can often be challenging. For example, it is not always trivial to physically interpret the state estimates in FIGS. 4B and 4C in a way that aligns with the process behavior exhibited in FIG. 4A. Nevertheless, it is often possible to ascribe mathematical interpretations to the results. In summary, an application of Algorithm 1 in quantifying and analyzing the behavior of a manipulated variable in a scale-up operation is provided. The developed tool can be general, however, and can be used in other related applications, such as scale-down model qualification, process characterization studies (see Tsang et al., 2014, Biotechnology progress, 30:152-160; Li et al., 2006, Biotechnology Progress, 22:696-703), comparisons of media formulations (see Jerums et al., 2005, BioProcess Int., 3:38-44; Wurm, Nature Biotechnology, 2004, at 22:1393), and mixing efficiencies in single-use and stainless steel bioreactors (see Eibl et al., 2010, Applied Microbiology and Biotechnology, 86:41-49; Diekmann et al., 2011, BMC Proceedings, 5: P103). These are important yet challenging problems in biopharmaceutical manufacturing, and the data-based scaling method disclosed herein can complement the existing knowledge-based solutions.

### Use Case 1, Example B: Small-Scale Model Qualification

Process characterization (PC) study is a key step in biopharmaceutical manufacturing for identifying critical process parameters (CPPs), material attributes, control strategy, and design space. See Godavarti et al., 2005, Biotechnology and Bioprocessing Series, 29:69. PC studies typically involve running multiple experiments on a commercial process with varied process conditions in order to identify the optimal design space. Since it is impractical, mainly due to economic considerations, to perform many PC assessments at the commercial-scale, PC studies are typically performed as a bench-scale process. To ensure that the performance of the bench-scale process is representative of the commercial process, it is generally important to first build a qualified bench-scale process, as inaccurate models often result in conclusions based on lab data that is not applicable to large-scale and therefore often leads to unsuccessful validation campaigns. See Varga et al., 2001, Biotechnology and Bioengineering, 74:96-107. A qualified scale-down model eliminates (or reduces) the need to conduct expensive experiments with the at-scale equipment. As a result, small-scale models find wide use in PC studies, process-fit studies, manufacturing troubleshooting, viral clearance studies, investigation of raw material variability, cell line selection studies, and process and media improvement studies. See FDA, 2011, “Guidance for industry. process validation: General principles and practices,” US Department of Health and Human Services, Rockville, MD, USA, vol. 1, pp. 1-22.

A typical cell-culture process involves several scales of operation, encompassing inoculum development and seed expansion up through production. To ensure that the small-scale and the commercial processes meet the same operating window, it is important to establish equivalency between the scales based on key performance parameters, such as: product quality; product titer; viable cell density (VCD); carbon dioxide profiles; pH profiles; osmolarity profiles; and metabolite profiles (e.g., glucose, lactate, glutamate, glutamine, ammonium). A fully qualified small-scale model and a commercial process are expected to exhibit similar profiles across all key performance parameters.

The qualification of a cell culture process is a challenging and time-consuming task that requires running multiple experiments on the small-scale bioreactor and careful design and tuning of the control parameters. Recently, the Process Validation Guidance report (FDA, 2011, “Guidance for industry. process validation: General principles and practices,” US Department of Health and Human Services, Rockville, MD, USA, vol. 1, pp. 1-22) released by the FDA states that “[i]t is important to understand the degree to which models represent the commercial process, including any differences that might exist, as this may have an impact on the relevance of information derived from the models.” In recent years, several statistical methods, such as equivalence testing of means and multivariate statistics, have been proposed to assess the quality of the small-scale model. The basic idea of equivalence testing is as follows: first, an a priori interval is defined within which the difference between the means of some key performance parameter at two scales (small-scale and commercial-scale) is assumed to be not practically meaningful. The difference of the means at two different scales is then evaluated using a two-one-sided-t-test (TOST), which calculates the confidence interval on the difference of means. The equivalency between the scales (with respect to the chosen performance parameter) is then established by comparing the confidence intervals obtained from TOST to the pre-defined intervals. See Li et al., 2006, Biotechnology Progress, 22:696-703. The equivalence testing of means is commonly used for validating key parameters, such as peak VCD, integrated VCD, final titer, and percentage of glycosylation isoform. Most of the performance parameters validated with TOST assume single-values instead of time-series. For example, it is not clear how TOST can be used to compare time-varying metabolite concentrations at different scales.

Current practices for comparing time-varying parameters include the use of qualitative methods. For example, the metabolite profiles at different scales are often compared using visual-based methods or through simple statistics, such as mean and variance. See Li et al., 2006, Biotechnology Progress, 22:696-703. Notwithstanding the simplicity of the visual-based methods, it is often challenging to quantify the degree of similarity (or dissimilarity) between the time-varying parameters. Alternatively, a multivariate statistical method for comparing time-varying parameters has been proposed. See Tsang et al., 2014, Biotechnology Progress, 30:152-160. The key idea is as follows: first, a partial least-squares (PLS) model is built for the parameters of the commercial process (e.g., VCD, glucose, lactate, glutamine, glutamate, ammonium, carbon dioxide, cell viability, pH, etc.) using historical data. Next, for the given PLS model, the parameters of the small-scale process is projected onto the model plane. If the small-scale model is fully qualified for the commercial process, then the projected data set can be explained by the PLS model; otherwise, there would be a divergence. In other words, a PLS model built for a commercial process can explain variations in the small-scale process, if and only if the small-scale process is fully qualified. This observation is valid for volume independent parameters, such as such as pH, dissolved oxygen, temperature, etc. However, for volume-dependent parameters, such as working volume, feed volume, agitation, and aeration, this is not necessarily true. This is because volume-dependent parameters scale according to the volume of the bioreactor. Furthermore, building a reliable PLS model for the commercial process requires access to large amounts of historical data (see Tulsyan et al., 2018, Biotechnology and Bioengineering, 115:1915-1924), and this requirement is contrary to the objective of a building a qualified small-scale model, i.e., to reduce the number of experiments on the commercial process. Finally, none of the existing methods quantify the degree of similarity, or lack thereof, in the performance parameters. As stated in the 2011 FDA guidance, understanding the degree to which the small-scale model represents the commercial process, allows one to better understand the relevance of information derived from the model.

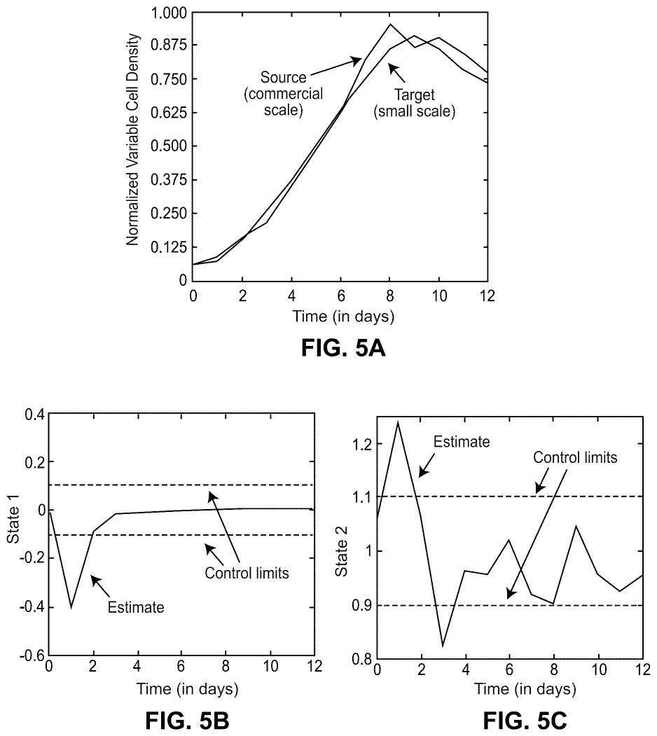

The efficacy of Algorithm 1 in comparing the time-varying parameters arising in small-scale model qualification studies is demonstrated. For illustration purposes, only the VCD profiles for a biologic produced in small-scale and commercial-scale bioreactors were compared. It is understood, however, the proposed method can be extended to compare other performance parameters as well. Formally, let {Xₜ}_{t∈N} and {Yₜ}_{t∈N} represent the mean VCD profiles in a commercial-scale and a small-scale bioreactor, respectively. FIG. 5A illustrates the normalized VCD profiles for a biologic produced in a 2000 liter commercial-scale bioreactor (here, the “source” process) and a 2 liter small-scale bioreactor (here, the “target” process). The mean profiles in FIG. 5A are calculated by averaging the VCD profiles over multiple small-scale and commercial-scale runs. Given the profiles in FIG. 5A, the objective is to quantify how similar (or dissimilar) the profiles are at each sample time. As discussed above, the traditional methods for comparing time-varying performance parameter in FIG. 5A are based on either “visual” inspection or the use of elementary process-knowledge, both of which are sub-optimal methods and do not quantify the degree of similarity. To address the limitations with existing methods, the AD ASTRA application 130 (e.g., the scaling model generation unit 140) can, in some embodiments, use Algorithm 1 to compare {Xₜ}_{t∈N} and {Yₜ}_{t∈N} continuously, and in real-time. First, it is assumed that {Xₜ}_{t∈N} and {Yₜ}_{t∈N} are related according to the SSM of Equations 6a and 6b, with

$${A}_{t} = [ \begin{matrix}0.6 & 0 \\ 0 & 1\end{matrix} ] , \qquad ( Equation 17 a )$$

$${Q}_{t} = {10}^{- 3} [ \begin{matrix}1 & 0 \\ 0 & 1\end{matrix} ] , \qquad ( Equation 17 b )$$

$${R}_{t} = {10}^{- 3} , \qquad ( Equation 17 c )$$

for all t∈ [symbol] . The eigenvalues of the system matrix, Aₜ, in Equation 17a describe stabilizing dynamics for [symbol] and random walk dynamics for [symbol] . Physically, for the choice of Aₜ in Equation 17a, the state sequence, [symbol] , goes to zero as t→∞, while the differences (if any) between the signals are captured by the state sequence, [symbol] . Next, the scaling model generation unit 140 can use Algorithm 1 to estimate θₜ=[θ_{1,t}, θ_{2,t}]^{T} for all t∈ [symbol] , with initial density, θ₀~ [symbol] (θ_{0|0}, P_{0|0}), such that

$${θ}_{0 \vert 0} = [ \begin{matrix}0 \\ 0\end{matrix} ] , \qquad ( Equation 18 a )$$

$${P}_{0 \vert 0} = [ \begin{matrix}1 & 0 \\ 0 & 1\end{matrix} ] , \qquad ( Equation 18 b )$$

FIGS. 5B and 5C give point-wise estimates of the states, [symbol] and [symbol] , respectively as calculated using Algorithm 1. As can be seen in FIGS. 5B and 5C, the estimates are time-varying rather than constant, and thus indicate non-uniform scaling between the VCD profiles in FIG. 5A. Mathematically, for the choice of the scaling model in Equation 5b, the signals {Xₜ}_{t∈N} and {Yₜ}_{t∈N} are equal if and only if [symbol] =[0, 1]^{T}. As expected, [symbol] in FIG. 5B converges to zero after Day 3. The non-zero values for [symbol] in FIG. 5C indicate a multiplicative relation between {Xₜ}_{t∈N} and {Yₜ}_{t∈N}. FIGS. 5B and 5C represent the estimated scaling factors (“Estimate”) calculated using Algorithm 1. Together, FIGS. 5B and 5C quantify and highlight the regions of similarity and dissimilarity between the VCD profiles in FIG. 5A. Finally, the dashed lines in FIGS. 5B and 5C represent the upper and lower control limits for the scaling factors. The control limits may be defined by engineers based on the requirements set for the small-scale model. For example, for the control limits set in FIGS. 5B and 5C, the VCD profiles in FIG. 5A can be assumed to be similar, except on Days 1 and 3, where States 1 and 2 are outside the control limits. Based on this assessment, if required, the engineers can further fine-tune their small-scale model for Days 1 and 3. Notably, FIGS. 5B and 5C are unique to the scaling model defined in Equations 17a-17c. Changing the system parameters in Equations 17a-17c or 18a-18b defines a new model, and therefore yields different state estimates. Nevertheless, for a given model, the estimates obtained with Algorithm 1 are guaranteed to be optimal (in terms of MSE).

In summary, an application of Algorithm 1 in small-scale model qualification of a cell-culture process has been demonstrated. Again, the developed tool can be general, and can be used in other related applications, such as process scale-up studies (see Junker, 2004, Journal of Bioscience and Bioengineering, 97:347-364; and Xing et al., 2009, Biotechnology and Bioengineering, 103:733-746), comparisons of media formulations (Jerums et al., 2005, BioProcess Int., 3:38-44; and Wurm, 2004, Nature Biotechnology, 22:1393), and mixing efficiencies in single-use and stainless steel bioreactors (Eibl et al., 2010, Applied Microbiology and Biotechnology, 86:41-49; and Diekmann et al., 2011, BMC Proceedings, 5: P103).

### Use Case 2: Comparing Multiple Signals

The developments in the previous section are generalized here to include multiple signals. Formally, a given target signal is compared against M source signals. Many problems in industrial biopharmaceutical manufacturing can be reformulated and cast into problems that require comparing multiple signals. The problem is formally defined below.

Let {X_{i,t}}_{t∈N} for all i=1, 2, . . . , M denote a set of M∈ [symbol] source signals and let {Yₜ}_{t∈N} denote a target signal. It is assumed that the M source signals are independently generated. Now, given a set of M source signals and a target signal, the objective is two-fold: first, to compare the target signal to the M source signals, and second, to rank the M source signals based on how similar they are to the target signal. The AD ASTRA application 130 (e.g., scaling model generation unit 140) can again use Algorithm 1 for pair-wise comparison of the target and source signals. For example, using Algorithm 1, the posterior density for the scaling factors between any signal pair, denoted generically as (X_{i,1:t}=X_{i,1:t}, Y_{1:t}=y_{1:t}), is given as

$$p ( {θ}_{1 , t} \vert {y}_{1 : t} ) = \text{?} ( {θ}_{i , t} \vert {μ}_{i , t} , {Σ}_{i , t} ) \qquad ( Equation 19 )$$

for all t∈ [symbol] , where μ_{i,t} and Σ_{i,t} are the mean and covariance of θ_{i,t}, respectively. Given M independent source signals, Algorithm 1 can be applied to each pair (x_{i,1:T}, y_{1:T}) to generate (μ_{i,1:T}, Σ_{i,i:T}) for all i=1, 2, . . . , M. Finally, for each i=1, 2, . . . , M, the signal pair (x_{i,1:T}, y_{1:T}) can be compared purely in terms of their scaling factors, θ_{i,1:T}, as discussed above.

The next objective is to rank the source signals, x_{i,1:T}, for all i=1, 2, . . . , M based on how similar the signals are to the target, y_{1:T}. A naive approach to rank source signals closest to the target is based on the Euclidean distance. For example, the Euclidean distance between (x_{i,1:T}, y_{1:T}) is given as follows

$${D}_{E} ( {x}_{i , 1 : T} , {y}_{1 : T} ) = \sqrt{{\sum }_{t = 1}^{T} {( {x}_{i , t} - {y}_{t} )}^{2}} , \qquad ( Equation 20 )$$

for all i=1, 2, . . . , M, where D_{E}(⋅,⋅) is the Euclidean distance. Based on the metric in Equation 20, the pair of signals with the smallest D_{E} value can also be regarded as the most similar. The Euclidean distance is relatively simple to implement, but it suffers from several drawbacks. First, in high-dimensional spaces, Euclidean distances are known to be unreliable. See Zimek et al., 2012, Statistical Analysis and Data Mining: The ASA Data Science Journal, 5:363-387. For example, in Equation 20, the signals are in [symbol] , and for large T values and in the presence of low signal-to-noise ratio, the calculation in Equation 20 may be unreliable. To circumvent the problems with the Euclidean distance, the AD ASTRA application 130 can instead use Kullback-Leibler divergence (KLD) to rank the signals. Unlike the Euclidean distance, the KLD works in a probability space. For example, for any two continuous random variables, P~p(z) and Q~q(z), the KLD between them is

$${D}_{KL} ( p \vert \vert q ) = {\int }_{- ∞}^{+ ∞} p ( z ) log [ \frac{p ( z )}{q ( z )} ] dz \qquad ( Equation 21 )$$

In machine learning literature, D_{KL}(p∥q) is called the “information gain” if p is used instead of q. Conversely, if q is a probability density function (PDF) of the source signal and p is a PDF of the target signal, then Equation 21 is the amount of “information lost” when q is used to approximate p. Therefore, in terms of KLD, the smaller the information loss, the less dissimilar (in probability) p and q are. The dissimilarity in KLD is different from dissimilarity in the Euclidean, as signals can be more dissimilar in the Euclidean but less dissimilar in the KLD. Finally, the KLD is an unbounded metric; it varies from 0 (for least divergence between PDFs) to +1 (for most divergence between PDFs). Further still, the KLD is a measure of divergence rather [symbol] than similarity. To bound and convert the KLD into a measure of similarity, one can define a KL convergence (KLC), [symbol] which is given as in Nowakowska et al., 2014, “Tractable Measure of Component Overlap for Gaussian Mixture Models”, arXiv: 1407.7172:

$${\text{?}}_{KL} ( p \vert \vert q ) = \frac{1}{1 + {D}_{KL} ( p \vert \vert q )} \qquad ( Equation 22 )$$

For any two PDFs, p and q, we have 0≤ [symbol] ≤1, where [symbol] =0 (or D_{KL}=+∞) represents least similar PDFs and [symbol] =1 (or D_{KL}=0) represents most similar PDFs. Notably, the KLD (or KLC) does not lend itself to a closed-form solution for arbitrary PDFs. For multivariate Gaussian densities, however, Equation 21 can be analytically solved.

Letting P and Q be two d-dimensional multivariate Gaussian variables distributed according to P~ [symbol] (μₚ, Σₚ), respectively, the KLD measure between P and Q (denoted by D_{KL}(p∥q)) is given as

$${D}_{KL} ( P \vert \vert Q ) = \frac{1}{2} [ log \frac{\vert {Σ}_{q} \vert}{\vert {Σ}_{p} \vert} + Tr [ {Σ}_{q}^{- 1} {Σ}_{p} ] + {[ {μ}_{q} - {μ}_{p} ]}^{T} {Σ}_{q}^{- 1} [ {μ}_{q} - {μ}_{p} ] - d ] . \qquad ( Equation 23 )$$

To be able to use this to rank source signals, the target and source signals need to be Gaussian distributed. Even if it is assumed that the signals are Gaussian, the sufficient statistics (i.e., mean and the covariance) for the signals are seldom available in practical settings. Further, computing an estimate based on a single sample trajectory is also challenging, unless the signal is independent and identically distributed (in which case the mean and covariance are stationary). In other words, direct calculation of the KLD (or KLC) between the source and target signals is not feasible under current settings and assumptions. Instead of computing the KLD between the source and target signals, therefore, computing the KLD for the scaling factors between the source and target signals may be implemented. This is plausible as the scaling factors in Equation 19 follow a multivariate Gaussian distribution with mean and covariance as given by Algorithm 1. Using the proposed method, the source signals can be ranked as follows: first, for the choice of an arbitrarily target signal, \tilde{y}_{1:T}, let θ_{*,t}∥\tilde{y}_{1:t}~p(θ_{*,t}|\tilde{y}_{1:t}) and θ_{j,t}|\tilde{y}_{1:t}~p(θ_{j,t}|\tilde{y}_{1:t}) for all t=1, . . . , T denote the scaling factors between (y_{i:T}, \tilde{y}_{1:T}) and (x_{j,1:T}, \tilde{y}_{1:T}), respectively, as calculated using Algorithm 1. Now, since p(θ_{*,t}|\tilde{y}_{1:t}) and p(θ_{j,t}|\tilde{y}_{1:t}) are both multivariate Gaussian distributions for all t=1, . . . , T, the KLD between the PDFs can be calculated using Equation 23. In fact, the KLD between p(θ_{*,t}|\tilde{y}_{1:t}) and p(θ_{j,t}|\tilde{y}_{1:t}) for all t=1, . . . , T, and j=1, . . . , M can be obtained likewise.

Assuming p(θ_{k,t}|\tilde{y}_{1:t}) and p(θ_{*,t}|\tilde{y}_{1:t}) yield the smallest KLD for some k∈{1, 2, . . . , M}, then the similarity in the scaling factors between (yₜ, \tilde{y}ₜ) and (x_{k,t}, \tilde{y}ₜ) implies similarity between yₜ and x_{k,t}. This claim is best understood by revisiting Equation 19. For the pair (x_{k,t}, \tilde{y}ₜ), the posterior PDF for the scaling factors at time t can be alternatively written as

$$p ( {θ}_{k , t} \vert {\tilde{y}}_{1 : t} ) ≡ p ( {θ}_{k , t} \vert {\tilde{y}}_{1 : t} , {A}_{t} , {R}_{t} , {Q}_{t} , {C}_{k , t} = [ 1 , {x}_{k , t} ] , {θ}_{0 \vert 0} , {P}_{0 \vert 0} ) , \qquad ( Equation 24 )$$

where the right-hand-side in Equation 24 explicitly lists all the parameters of the scaling model, noise statistics, and the initial density that the posterior density actually depends on. Similarly, for the pair (yₜ, \tilde{y}ₜ), the posterior density can be written as

$$p ( {θ}_{* , t} \vert {\tilde{y}}_{1 : t} ) ≡ p ( {θ}_{* , t} \vert {\tilde{y}}_{1 : t} , {A}_{t} , {R}_{t} , {Q}_{t} , {C}_{* , t} = [ 1 , {y}_{t} ] , {θ}_{0 \vert 0} , {P}_{0 \vert 0} ) . \qquad ( Equation 25 )$$

If the parameter set (Aₜ, Rₜ, Qₜ, θ_{0|0}, P_{0|0}) in Equations 24 and 25 is the same, then from the uniqueness of the Kalman filter solution p(θ_{*,t}|\tilde{y}_{1:t})=p(θ_{k,t}, \tilde{y}_{1:t}) implies yₜ=x_{k,t}. In other words, similarity between the PDFs p(θ_{*,t}|\tilde{y}_{1:t}) and p(θ_{k,t}|\tilde{y}_{1:t}) implies similarity between yₜ and x_{k,t}. Notably, the similarity between the signals yₜ and x_{k,t} is in the sense that conditioning Equation 24 over C=_{*,t} or C_{k,t} does not add any new information in the posterior calculations. Finally, the pseudo-code for the proposed signal ranking algorithm is outlined in Algorithm 2. In Algorithm 2, the choice of \tilde{y}_{1:T} can be arbitrary. For example, it is possible to choose \tilde{y}ₜ=yₜ for all t=1, . . . , T.

Algorithm 2, which may be implemented by the AD ASTRA application 130 in some embodiments, is as follows

Algorithm 2: Signal Ranking

| | |
|---|---|
| 1. | $\begin{matrix}Input : Scaling model and signals : {A}_{t} , {R}_{t} , {Q}_{t} , {θ}_{0 \vert 0} , {P}_{0 \vert 0} , \\ {y}_{1 : T} , {\{ {x}_{i , 1 : T} \}}_{i = 1}^{M} and {\tilde{y}}_{1 : T}\end{matrix}$ |
| 2. | Output: Index set: index ∈ {1, 2, . . . , M}^{M} with unique entries, |
| | such that index[1] and index[M] denoting the indices of the |
| | source signals that are most and least similar to the target |
| | signal, respectively. |
| 3. | $Compute {\{ p ( {θ}_{* , t} \vert {\tilde{y}}_{1 : t} ) \}}_{t = 1}^{T} for \{ {y}_{1 : T} , {\tilde{y}}_{1 : T} \} using Algorithm 1.$ |
| 4. | for i = 1 to M do |
| 5. | $Compute {\{ p ( {θ}_{i , t} \vert {\tilde{y}}_{1 : 𝔱} ) \}}_{𝔱 = 1}^{T} for \{ {x}_{i , 1 : T} , {\tilde{y}}_{1 : T} \} using Algorithm 1.$ |
| 6. | $Set \text{?} ← 0.$ |
| 7. | for t = 1 to T do |
| 8. | $\begin{matrix}Calculate \text{?} between p ( {θ}_{i , t} \vert {\tilde{y}}_{1 : t} ) and \\ p ( {θ}_{i , t} \vert {\tilde{y}}_{1 : t} ) using ( 23 ) and ( 22 ) .\end{matrix}$ |
| 9. | $Set \text{?} ← \text{?} + \text{?} .$ |
| 10. | end for |
| 11. | end for |
| 12. | $index ← Sort ( [ \text{?} , … , \text{?} ] {,}^{′} {ascend}^{′} ) .$ |

Other similarity measures can be used in place of (or in addition to) the KLD measure to rank the source signals, such as Weitzman's measure (Weitzman, 1970, US Bureau of the Census, vol. 22), Matusita's measure (Matusita, 1955, Annals of Mathematical Statistics, pp. 631-640), or Morisita's measure (Morisita, 1959, Mem. Fac. Sci. Kyushu Univ. Series E, 3-65-80). For example, the Weitzman's measure calculates the overlap between the two PDFs, where higher overlap corresponds to more similar PDFs. Mathematically, the Weitzman's measure, σ_{W}, is given as follows:

$$\text{?} ( p \vert \vert q ) = \int min \{ p ( x ) , q ( x ) \} dx , \qquad ( Equation 26 )$$

where p and q are two arbitrary PDFs. A procedure to calculate Equation 26 for univariate Gaussian densities is given in Inman et al., 1989, Communications in Statistics—Theory and Methods, 18:3851-3874. However, it is not straightforward to extend this to the multivariate case. For multivariate PDFs, Equation 26 can be calculated using Monte-Carlo (MC) methods, such as importance sampling (see Tulsyan et al., 2016, Computers & Chemical Engineering, 95:130-145). Notably, Equation 26 can be rewritten as

$$\text{?} ( p \vert \vert q ) = \int \frac{min \{ p ( x ) , q ( x ) \}}{r ( x )} r ( x ) dx , \qquad ( Equation 27 )$$

where r≡wp+(1−w)q is an importance PDF for some convex weight 0≤w≤1. It can be seen that supp(r)=supp(p)∪supp(q). Now, for a multivariate Gaussian densities p and q, r is a multivariate Gaussian mixture density.

$$If {\{ Z = {z}^{( i )} \}}_{i = 1}^{N}$$

represents a set of N random i.i.d. (independent and identically distributed) samples distributed according to r (note that random sampling from a mixture Gaussian PDF is well-established), then an MC estimate of Equation 27, denoted as σ_{W}, is given as

$$\text{?} ( p \vert \vert q ) = \frac{1}{N} {\sum }_{i = 1}^{N} \frac{min \{ p ( {z}^{( i )} ) , q ( {z}^{( i )} ) \}}{r ( {x}^{( i )} )} \qquad ( Equation 28 )$$

As with the KLD measure, the source signals can be ranked based on the Weitzman's measure. This is done by replacing the KLD measure in Algorithm 2 with the Weitzman's measure in Equation 28. However, since Equations 22 and 28 are two separate similarity measures, the rankings of source signals may vary. The framework described herein for comparing and ranking signals based on similarity is generic, and can be used to address several challenging problems in biopharmaceutical manufacturing that lend themselves to reformulations that require comparing and ranking signals. For example, in Trunflo et al., the authors considered the problem of placing purchase orders for mammalian cell culture raw materials that meet biologic production requirements. See Trunflo et al., 2017, Biotechnology Progress, 33:1127-1138. The authors proposed a chemometric model that compares spectroscopic scans of raw materials obtained from multiple vendors against the nominal material lot. The order is placed with the vendor, whose raw material scan is most similar to the nominal lot. While Trunflo et al. uses a chemometric model for comparing spectroscopic scans to the nominal scan, the AD ASTRA application 130 can do the same using Algorithm 2. In fact, an advantage of Algorithm 2 over chemometric methods, as in Trunflo et al., is that Algorithm 2 does not require a model for the nominal lot. This reduces or eliminates the need to collect a large amount of historical scans for the nominal lot. As an example, the problem of ranking bio-therapeutic proteins in a portfolio of products produced in commercial bioreactors based on their oxygen uptake profiles is considered. In general, this is an important class of problems in biopharmaceutical manufacturing, as comparing key process variables across multiple products helps improve basic understanding of the process dynamics of different products, and also in designing strategies for controlling process parameters that are similar across different products. For example, if two biologics have similar oxygen uptake profiles, their cell growth profiles can be expected to be similar. Furthermore, having knowledge of products with similar growth profiles allows engineers to deploy similar strategies for controlling the processes. The analysis and ranking of proteins using Algorithm 2 is discussed next.

### Use Case 2, Example A: Comparing Multiple Products

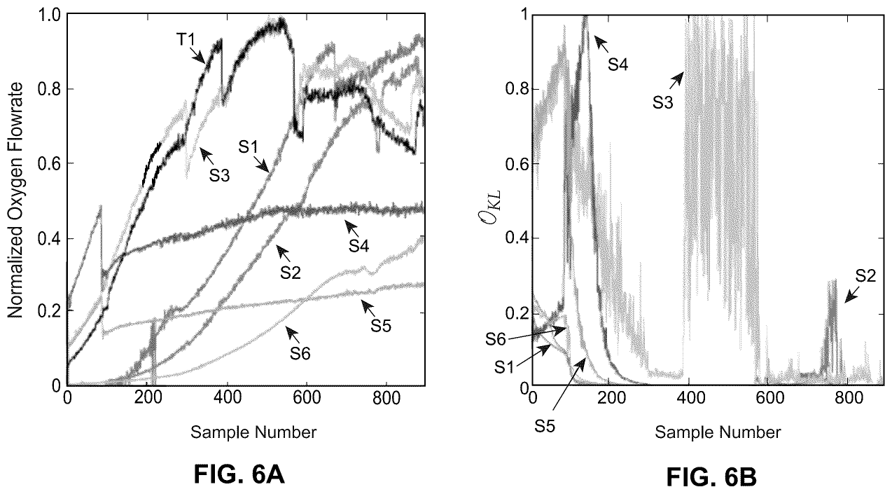

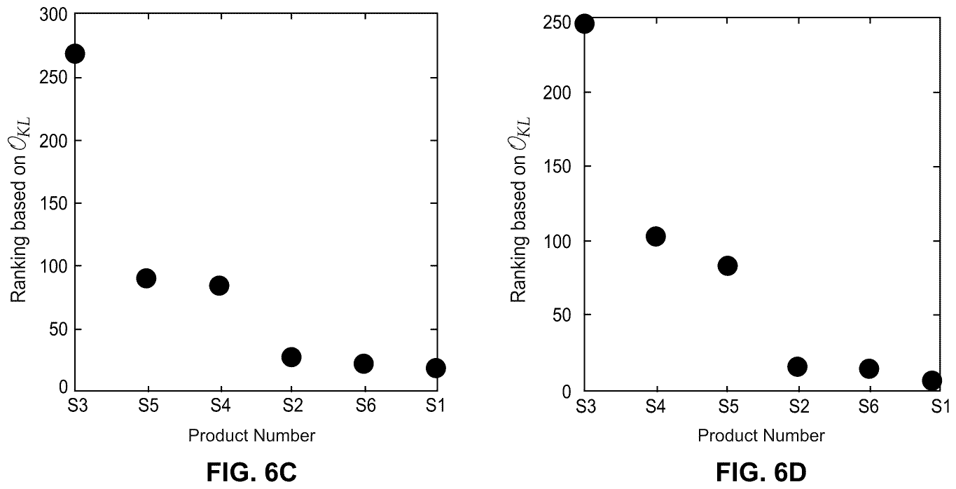

Next, the problem of comparing oxygen flow rate profiles for different biologics produced in commercial bioreactors, and ranking the biologics based on how their oxygen uptake profiles compare to that of a reference biologic, is considered. For example, FIG. 6A shows the normalized oxygen flow rate profiles for seven bio-therapeutic proteins produced in a commercial bioreactor. From FIG. 6A, it is clear that different biologics can have very different oxygen uptake requirements. Of the seven profiles shown in FIG. 6A, six of them (S1, S2, S3, S4, S5, S6) are for the “source” biologics, and the other (T1) is for the “target” biologic. Note that the distinction between the source and target biologics is strictly mathematical and decided based on the problem setting. In this example, we consider the following: given all the profiles in FIG. 6A, the objective is to find the profile in the set (S1, S2, S3, S4, S5, S6) that is most similar to T1, or more generally, rank the profiles in (S1, S2, S3, S4, S5, S6) based on their similarity to T1. This is an important problem, as oxygen uptake is a critical variable for controlling the level of dissolved oxygen in a bioreactor, and comparing the profiles across different products allow process engineers to better understand and control cell-growth profiles. To compare and rank the oxygen flow rate profiles in FIG. 6A using Algorithm 2, the source and target profiles are denoted as, x_{i,1:T}, where i=1, . . . , M, and y_{1:T}, respectively. Next, a dummy target signal, \tilde{y}_{1:T}, is also generated randomly. Here, it is assumed that M=6 and T=900. As outlined in Algorithm 2, using Algorithm 1 the scaling factors between (y_{1:T}, \tilde{y}_{1:T}) is calculated for the following scaling model:

$${A}_{t} = [ \begin{matrix}1 & 0 \\ 0 & 1\end{matrix} ] , \qquad ( Equation 29 a )$$

$${Q}_{t} = {10}^{- 2} [ \begin{matrix}1 & 0 \\ 0 & 1\end{matrix} ] , \qquad ( Equation 29 b )$$

$${R}_{t} = 0.1 \qquad ( Equation 29 c )$$

The initial density θ₀~ [symbol] (θ_{0|0}, P_{0|0}) in Algorithm 1 is a multivariate Gaussian density with mean and covariance given as

$${θ}_{0 \vert 0} = [ \begin{matrix}0 \\ 0\end{matrix} ] , \qquad ( Equation 30 a )$$

$${P}_{0 \vert 0} = [ \begin{matrix}1 & 0 \\ 0 & 1\end{matrix} ] \qquad ( Equation 30 b )$$

Again, using the model in Equations 29a-29c and 30a-30b, the scaling between (x_{i,1:T}, \tilde{y}_{1:T}) is calculated for all i=1, . . . , M. Once the posterior PDFs for the scaling factors are available, the KLC between the PDFs can be calculated, as outlined in Algorithm 2.

FIG. 6B gives the [symbol] measure calculated between the posterior PDFs: p(θ_{*,t}|\tilde{y}_{1:t}) and p(θ_{i,t}|\tilde{y}_{1:t}) for all t=1, . . . , T and i=1, . . . . , M. [symbol] varies not only across the product but also along the length of the campaign. For example, of all the six source signals, S3, S4, and S5 exhibit the highest [symbol] values in the interval 1≤t≤200, after which the values for S4 and S5 plummet in the interval 200<t≤900. FIG. 6C ranks the source profiles, S1, S2, S3, S4, S5, and S6 based on their similarity to T1 (as measured by [symbol] ). From FIG. 6C, it is evident that S3 is most similar to T1, and S1 is least similar to T1. Physically, the similarity between S3 and T1 is not surprising, as S3 is for a source biologic that is a high-titer version of the target biologic. Similarly, the dissimilarity between T1 and S1 is also expected, as T1 is produced in a 15,000 liter fed-batch bioreactor, whereas S1 is produced in a 2,000 liter perfusion bioreactor. This demonstrates the efficacy of the proposed method in accurately ranking the profiles, without any a priori information about the product or the process. Compared to S3, the second most similar product, S5, is significantly less similar to T1, such that it is not relevant for practical purposes. This is also evident in FIG. 6A, where the differences between S5 and T1 are clear. The results in FIG. 6C are based on uniform summation of [symbol] over the entire length of the campaign (see Step 9 in Algorithm 2). If the campaign operations at certain time intervals are more relevant than others, then it is possible to consider a weighted summation of σ_{KL}. This can be done by replacing Step 9 in Algorithm 2 with

$${\text{?}}_{, Total} ← {\text{?}}_{: , Total} + {ζ}_{t} \text{?} , \qquad ( Equation 31 )$$

where 0≤ζₜ≤1 is a positive weight. For the sake of brevity, the results based on weighted [symbol] are not shown here. However, the profile ranking based on Equation 31 can yield different results.

It is also possible to rank the profiles using the Weitzmann's measure, [symbol] , as opposed to the [symbol] measure in FIG. 6C. The profile ranking using [symbol] is shown in FIG. 6D. Similar to [symbol] , the measure [symbol] ranks S3 as the most similar to T1 an S1 as the least similar to T1. In fact, comparing FIGS. 6C and 6D, the rankings (relative order of similarity) suggested by [symbol] and [symbol] are identical, except for S4 and S5 which are flipped. In summary, the efficacy of Algorithm 2 is demonstrated in comparing and ranking multiple source signals based on their similarity to the reference target signal. Again, while the ranking of the oxygen flow rate profiles was considered, the techniques disclosed herein are generic, and can be used in other applications as well.

### Use Case 2, Example B: Monitoring Product Sieving

In biopharmaceutical manufacturing, recombinant proteins are commonly produced in batch or fed-batch bioreactors by culturing cells for two to three weeks to produce the protein of interest. As protein-based therapeutics continue to drive the demand for cheaper and higher volume production methods, continuous production options such as perfusion bioreactors are becoming a popular choice in industry. See Wang et al., 2017, Journal of Biotechnology, 246:52-60; and Pollock et al., 2013, Biotechnology and Bioengineering, 110:206-219. Unlike batch or fed-batch, perfusion bioreactors culture cells over much longer periods by continuously feeding the cells with fresh media and removing spent media while keeping cells in the culture. In addition to protein being continuously removed before being exposed to excessive waste that causes degradation, perfusion bioreactors offer several advantages over conventional batch processes, such as superior product quality, stability, scalability, and cost-savings. See Wang et al., 2017, Journal of Biotechnology, 246:52-60.

Tangential flow filtration (TFF) and alternating tangential flow (ATF) systems are commonly used for product recovery in perfusion systems. TFF operations continuously pump feed from the bioreactor across a filter channel and back to the bioreactor, while cell-free permeate is drawn off and collected. ATF systems use an alternating flow diaphragm pump that pulls and pushes feed from and to the bioreactor while cell-free permeate is drawn off. See Hadpe et al., 2017, Journal of Chemical Technology and Biotechnology, 92:732-740. A cell retention device is at the center of any perfusion system as it often relates to scalability, reliability, cell viability, and efficiency in terms of cell clarification at desired cell densities and product recovery. See Wang et al., 2017, Journal of Biotechnology, 246:52-60. In industry, hollow fiber membranes are the most preferred technology for cell retention, as they satisfy many of the aforementioned considerations. See Clincke et al., 2013, Biotechnology Progress, 29:754-767. Despite their wide use, hollow fiber filtration systems are susceptible to product sieving and membrane fouling. See Mercille et al., 1994, Biotechnology and Bioengineering, 43:833-846. Membrane fouling is a critical issue in any perfusion system as it generally results in ineffective product recovery across the membrane and gradual decrease of permeate over time, which can end a run prematurely. See Wang et al., 2017, Journal of Biotechnology, 246:52-60.

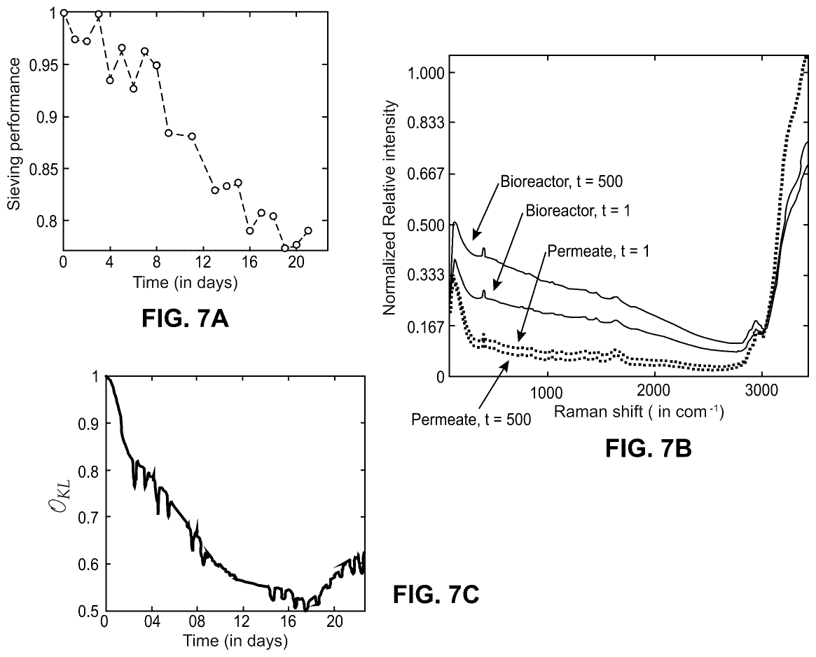

In practice, product sieving across the hollow fiber is defined as the ratio of protein concentration in the permeate line to protein concentration in the bioreactor. A 100% level of product sieving indicates total product passage across the membrane, and a 0% level of product sieving indicates zero product recovery. Mathematically, if h_{t,p}∈ [symbol] and h_{t,b}∈ [symbol] represent protein concentrations in the permeate and bioreactor, respectively, then product sieving across the hollow fiber, yₜ∈ [symbol] , is calculated as

$${γ}_{t} = \frac{{h}_{t , p}}{{h}_{t , b}} , \qquad ( Equation 32 )$$

where 0≤γₜ≤1 for all t∈N. FIG. 7A shows the sieving profile for a biotherapeutic protein produced in a 50 liter perfusion bioreactor fitted with an ATF. The sieving performance is calculated using Equation 32 based on offline titer measurements from the bioreactor and permeate. The titer samples were collected once daily from the bioreactor and the permeate line at the same time point, and analyzed using a Cedex BioHT for monoclonal antibody concentration. The time axis in FIG. 7A is scaled such that Day 0 corresponds to the start of product harvest. The performance in FIG. 7A is also scaled to ensure that the membrane delivers 100% product sieving at Day 0, i.e., γ₀=1. Starting at γ₀=1, it can be seen in FIG. 7A that the sieving performance of the ATF reduces over time due to fouling.

Although the model of Equation 32 is commonly used in practice for assessing sieving performance, it provides limited resolution. For example, much of the intra-day product sieving information in FIG. 7A is unavailable. This is because the current technology for real-time titer measurements or product sieving in Equation 32 is either unreliable or too expensive. One approach to deal with limited titer measurements is to use Raman-based chemometric models. A partial least squares (PLS) model has been used to correlate Raman spectra to protein concentration in cell culture. Andre et al., 2015, Analytica Chimica Acta, 892:148-152. Once the PLS model is available, protein concentration can be predicted in-line using fast-sampled spectral data. While a chemometric model improves the resolution of the sieving profile, building a PLS model is a tedious task that requires access to large historical data sets. Further, the quality of predictions is both process dependent and media concentration dependent. While these efforts may be used for real-time applications, such as for closed-loop titer control in cell-culture (see Matthews et al., 2016, Biotechnology and Bioengineering, 113:2416-2424), a chemometric model might not be necessary for assessing membrane fouling. In this section, an alternative approach for real-time monitoring of product sieving across the hollow fiber, which may be implemented by the AD ASTRA application 130 by operating directly on the Raman spectra and using Algorithm 2, is provided.

First, in this example, a 50 liter perfusion bioreactor was fitted with two Raman spectroscopy probes, with one in the bioreactor and one in the permeate line. The Raman probes used were immersion type probes constructed of stainless steel. The probes were connected to a RamanRXN3 (Kaiser Optical Systems, Inc.) Raman spectroscopy system/instrument. A laser provided optical excitation at 785 nm resulting in approximately 200 mW of power at the output of each probe. Excitation in the far red region of the visible spectrum resulted in fluorescence signals from culture and permeate components. Each Raman spectrum was collected using a 75 second exposure time with 10 accumulations. Dark spectrum subtraction and a cosmic ray filter were also employed. The Raman spectra were measured every 15 minutes. FIG. 7B shows the Raman spectra collected from the bioreactor and the permeate at two different times, with normalized relative intensity values. Note that in FIG. 7B, any differences (in the Euclidean sense) in the bioreactor and permeate spectra at a given time are due to differences in the protein and metabolite concentrations across the hollow fiber membrane.

Next, instead of tracking changes in protein concentrations using a chemometric model, changes in Raman spectra were tracked. Since a Raman spectral signal implicitly includes/represents titer information, tracking spectral signals directly can yield information about membrane fouling (as it is a function of titer, see Equation 32). The AD ASTRA application 130 can perform this using Algorithm 2, as follows. First, let

$${x}_{t , \underline{s} \overline{s}}$$

$$and$$

$${y}_{t , \underline{s} \overline{s}}$$

represent spectral signals from the bioreactor and permeate, respectively, at time t and for Raman shifts, k=s . . . , s̅, where s=100 cm⁻¹ and s=3425 cm⁻¹. Let

$${θ}_{t , \underline{s} \overline{s}}$$

denote the scaling factor between

$${x}_{t , \underline{s} \overline{s}}$$

$$and$$

$${y}_{t , \underline{s} \overline{s}}$$

calculated using Algorithm 1. The sequence,

$${θ}_{t , \underline{s} \overline{s}}$$

summarizes the differences in media concentrations across the membrane. When there is no sieving loss, then the media concentrations across the membrane are the same and thus

$${x}_{t , \underline{s} \overline{s}} = {y}_{t , \underline{s} \overline{s}}$$

for all t∈N. Once fouling starts, however, the equality no longer holds and

$${θ}_{t , \underline{s} \overline{s}}$$

captures the differences between

$${x}_{t , \underline{s} \overline{s}}$$

$$and$$

$${y}_{t , \underline{s} \overline{s}}$$

Therefore, by tracking

$${θ}_{t , \underline{s} \overline{s}}$$

for all t∈N, one can assess the rate of membrane fouling.

Algorithm 2 provides an efficient way to track

$${θ}_{t , \underline{s} \overline{s}}$$

for all t∈N. Using Algorithm 2, the entries in

$$\{ {θ}_{t , \underline{s} \overline{s}} , … , {θ}_{T , \underline{s} \overline{s}} \}$$

are ranked with respect to

$${θ}_{t , \underline{s} \overline{s}} ,$$

where

$${θ}_{t , \underline{s} \overline{s}}$$

represents the state of the membrane at time t=1. Now, if p(θ_{1,m}|y_{1,s:m}) and p(θ_{j,m}|y_{j,s:m}) represent the posterior for the scaling factors at time t=1 and t=j, respectively, then similarity between the PDFs can be calculated using [symbol] (see Steps 4 through 11 in Algorithm 2). Physically, a larger [symbol] value represents more similar Raman spectra, which in turn implies similar media concentrations across the membrane. Conversely, with fouling, the spectral signal across the membrane will be different compared to that at t=1, thereby decreasing [symbol] .

FIG. 7C shows the [symbol] values as a function of time. FIG. 7C shows real-time product sieving information extracted directly from raw spectral data, without requiring any offline titer samples or chemometric models. Notably, unlike FIG. 7A where measurements are available only once per day, in FIG. 7C, measurements are available every 15 minutes. In fact, compared to FIG. 7A, FIG. 7C provides a much higher resolution. As seen in FIG. 7C, [symbol] rapidly decreases until Day 3 and then continues to decrease further until Day 17. This is because as titer increases in the bioreactor, stresses on the membrane also increase, thereby leading to higher pressure across the membrane. The rapid drop in [symbol] until Day 3 is indicative of a rapid rate of membrane degradation initially, followed by gradual degradation thereafter. After Day 17, the cells start producing less protein, leading to less membrane stress and therefore, higher [symbol] values.

FIGS. 7A and 7C present a complementary view on the product sieving problem. For example, while FIG. 7A presents instantaneous product sieving information, FIG. 7C indicates the rate of product sieving. This is because FIG. 7C uses the initial membrane state as the reference state. If the initial membrane state is altered, the results in FIG. 7C would also change accordingly. Also, while FIG. 7A is based on differences in titer concentrations, FIG. 7C is based on overall concentration differences, including titer and metabolite concentrations. This is because FIG. 7C uses Raman spectra, which encodes both titer and metabolite information. If desired, the effect of metabolite concentrations and/or other media constitutes can be mitigated by selecting regions of spectra that are sensitive to titer alone.

In summary, this highlights how the problem of monitoring product sieving can be reformulated as a problem that requires comparison of multiple signals, and how Algorithm 2 provides an effective practical solution to that problem. Again, while in this example Algorithm 2 was used to monitor product sieving, the developed method is generic and can be used in other applications that require comparison of multiple signals.

### Use Case 3: Data Projection

The problem of data projection is now considered, wherein the objective is to project the signals (or data sets) generated at one scale (e.g., pilot scale) to another scale (e.g., commercial scale), such that the projected signals are representative of the process at the new scale. Data projection is an important class of problem in biopharmaceutical manufacturing, as a typical life cycle of biologic production generates data across three different scales of operations, namely bench-top scale, pilot scale, and commercial scale. For such multi-scale process operations, projecting data sets from one scale to another scale allows for derivation of early critical process insights, data reuse and data recycling across different scales, and potential reduction in experiments needed at different scales. The problem of data projection can be reformulated and viewed as a data scaling problem, wherein the objective is to re-scale the signals generated at (size) Scale 1 to make them representative of the process behavior at (size) Scale 2. Signals at Scale 1 and Scale 2 will be referred to here as source and target signals, and denoted generically as

$${\{ {x}_{m , 1 : T} \}}_{m = 1}^{M} and {\{ {y}_{n , 1 : T} \}}_{n = 1}^{N} ,$$

respectively, where Tis the length of the signal, and M≥1 and N≥1 are the number of source and target signals, respectively. The condition N≥1 ensures that there is at least one target signal available, which enables the scaling model generation unit 140 to determine/generate the scaling model. The M source signals and the N target signals are assumed to span a source space and a target space, respectively. Further, for convenience, the source and target spaces are assumed to represent the same variable of interest, e.g., agitation or pH, although this is not necessarily the case in all embodiments and/or scenarios.

Given

$${\{ {x}_{m , 1 : T} \}}_{m = 1}^{M} and {\{ {y}_{n , 1 : T} \}}_{n = 1}^{N} ,$$

the objective is to estimate

$${\{ {y}_{m , 1 : T}^{*} \}}_{m = 1}^{M} ,$$

where

$${y}_{m , 1 : T}^{*}$$

is a projection of x_{m,1:T} onto the target space. To obtain the projections of source signals, a scaling model between the source and the target space is first defined (i.e., generated by the scaling model generation unit 140). Once a scaling model is defined/generated, the data conversion unit 142 can pass the source signals through the scaling model to obtain their projection on the target space. This is the central idea behind the proposed method for data projection, and is discussed in detail below.

One approach to generating a scaling model between the source space and the target space is to define the scaling model in terms of the signals. For example, for any pair of source-target signal, {x_{i,1:T}, y_{j,1:T}}, where (i,j)∈{1, . . . , M}×{1, . . . , N}, the signals are assumed to be related according to the following scaling model

$${y}_{j ; t} = {c}_{i , t} {θ}_{i , j , t} + {ϵ}_{i , j , t} , \qquad ( Equation 33 )$$

where c_{i,t}=[1, x_{i,t}]∈ [symbol] , θ_{i,j,t}∈ [symbol] is the scaling factor between the i-th source signal and j-th target signal, and

$${ϵ}_{i , j , t} ∼ \text{?} ( 0 , {σ}_{t}^{2} )$$

is the noise. In Equation 33, each pair, {x_{i,1:T}, y_{j,1:T}}, defines a unique scaling model. This is because of the inherent variability in the source and target signals due to sensor noise, batch-to-batch variability, and other known or unknown disturbances. To uniquely capture the relationship between the spaces, a scaling model is defined between the mean source signal and the mean target signal. Mathematically, if \tilde{x}_{1:T} and \tilde{y}_{1:T} denote the mean source signal and the mean target signal, respectively, then the signals are related as follows

$${\overline{y}}_{t} = {\overline{c}}_{t} {θ}_{t} + {ϵ}_{t} , \qquad ( Equation 34 )$$

where c̅ₜ=[1, x̅ₜ]∈ [symbol] , θₜ∈ [symbol] is the scaling factor and

$${ϵ}_{t} ∼ \text{?} ( 0 , {σ}_{t}^{2} )$$

is the noise. The model in Equation 34 defines the relationship between the source and target spaces in terms of expected signal profiles. Given {x̅_{1:T}, y̅_{1:T}}, the posterior density for θ_{1:T} can be estimated using Algorithm 1, such that

$$p ( {θ}_{t} \vert {\overline{y}}_{1 : t} ) = \text{?} ( {θ}_{t} \vert {θ}_{t \vert t} , {P}_{t \vert t} ) , \qquad ( Equation 35 )$$

where θ_{t|t} and P_{t|t} are the posterior mean and the posterior covariance, respectively. Next, using Equations 34 and 35, the source signal x_{m,1:T} (where m=1, . . . , M) can be projected onto the target space by replacing θₜ in Equation 34 with its point estimate, such that

$${y}_{m , t}^{*} = {c}_{m , t} {θ}_{t \vert t} \qquad ( Equation 36 )$$

where c_{m,t}=[1, x_{m,t}] and

$${y}_{m , t}^{*}$$

is a projection of x_{m,t} onto the target space, for all t=1, . . . , T. The projection in Equation 36 is scale-preserving in the sense that

$$\{ {x}_{m , 1 : T} , {y}_{m , 1 : T}^{*} \}$$

and {x̅_{1:T}, y̅_{1:T}} share the same scaling factors, i.e., θ_{1:T|1:T}. In other words, Equation 36 preserves the inherent differences between the source and target spaces. Note that while Equation 36 is scale preserving, it depends on the choice of the point estimate. Recall that the posterior density for the scaling factors is a Gaussian density, with mean θ_{t|t} and covariance P_{t|t}.

A Bayesian approach to project source signals onto the target space under uncertainty is to construct a posterior density,

$$p ( {y}_{m , t}^{*} \vert {\overline{x}}_{1 : t} , {\overline{y}}_{1 : t} , {x}_{m , 1 : t} ) ,$$

independent of the scaling factors. Notably,

$$p ( {y}_{m , t}^{*} \vert {\overline{x}}_{1 : t} , {\overline{y}}_{1 : t} , {x}_{m , 1 : t} )$$

only depends on the set {\tilde{x}_{1:t}, \tilde{y}_{1:t}, x_{m,1:t}}. Then, using the law of marginalization, one can rewrite the posterior density,

$$p ( {y}_{m , t}^{*} \vert {\overline{x}}_{1 : t} , {\overline{y}}_{1 : t} , {x}_{m , 1 : t} )$$

as

$$p ( {y}_{m , t}^{*} \vert {\overline{x}}_{1 : t} , {\overline{y}}_{1 : t} , {x}_{m , 1 : t} ) = \int p ( {y}_{m , t}^{*} , d {θ}_{t} \vert {\overline{x}}_{1 : t} , {\overline{y}}_{1 : t} , {x}_{m , 1 : t} ) , \qquad ( Equation 37 a )$$

where p(dθₜ|\tilde{x}_{1:t}, \tilde{y}_{1:t}, x_{m,1:t})=p(θₜ|\tilde{x}_{1:t}, \tilde{y}_{1:t}, x_{m,1:t})dθₜ.

In Equation 37a, the scaling factors are marginalized out:

$$p ( {y}_{m , t}^{*} \vert {\overline{x}}_{1 : t} , {\overline{y}}_{1 : t} , {x}_{m , 1 : t} ) = \int p ( {y}_{m , t}^{*} \vert {θ}_{t} , {x}_{m , t} ) p ( {θ}_{t} \vert {\overline{x}}_{1 : t} , {\overline{y}}_{1 : t} ) d {θ}_{t} , \qquad ( Equation 38 a )$$

$$= \int 𝒩 ( {y}_{m , t}^{*} \vert {c}_{m , t} {θ}_{t} , {σ}^{2} ) 𝒩 ( {θ}_{t} \vert {θ}_{t \vert t} , {P}_{t \vert t} ) d {θ}_{t} , \qquad ( Equation 38 b )$$

It can be shown that

$$p ( {y}_{m , t}^{*} \vert {\overline{x}}_{1 : t} , {\overline{y}}_{1 : t} , {x}_{m , 1 : t} )$$

in Equation 38b also follows a normal distribution with (see Särkkä, Simo, Bayesian Filtering and Smoothing, No. 3, Cambridge University Press, 2013):

$$p ( {y}_{m , t}^{*} \vert {\overline{x}}_{1 : t} , {\overline{y}}_{1 : t} , {x}_{m , 1 : t} ) = N ( {y}_{m , t}^{*} \vert {c}_{m , t} {θ}_{t \vert t} , {c}_{m , t} {P}_{t \vert t} {c}_{m , t}^{T} + {σ}^{2} ) \qquad ( Equation 39 )$$

Equation 39 gives the entire distribution of projection of the source signal onto the target space. Note that Equation 39 is independent of any specific realization of the scaling factors. The mean of the posterior density in Equation 39 is

$$E ( {y}_{m , t}^{*} \vert {\overline{x}}_{1 : t} , {\overline{y}}_{1 : t} , {x}_{m , 1 : t} ) = {c}_{m , t} {θ}_{t \vert t}$$

and variance is

$$V ( {y}_{m , t}^{*} \vert {\overline{x}}_{1 : t} , {\overline{y}}_{1 : t} , {x}_{m , 1 : t} ) = {c}_{m , t} {P}_{t \vert t} {c}_{m , t}^{T} + {σ}^{2} .$$

Statistically, any single random realization from Equation 39 can be regarded as a potential projection of x_{m,t} onto the target space. Alternatively, it is a common practice to assume the mean of the distribution as the point-estimate such that:

$${y}_{m , t}^{*} = E ( {y}_{m , t}^{*} \vert {\overline{x}}_{1 : t} , {\overline{y}}_{1 : t} , {x}_{m , 1 : t} ) = {c}_{m , t} {θ}_{t \vert t} \qquad ( Equation 40 )$$

Comparing Equation 40 and Equation 36, the Bayesian approach and the frequentist approach both yield the same point-estimate for the projection of x_{m,t} onto the target space; however, note that with the Bayesian approach it is also possible to ascribe quality to the point estimate in Equation 40. This can be done using the variance of the posterior density in Equation 39.

Finally, Algorithm 3 gives the outline of how proposed method can be used to project signals from source to target spaces. Algorithm 3 is as follows:

Algorithm 3 - Projecting Signals

| | |
|---|---|
| 1. | $\begin{matrix}Input : Scaling model and signals : \\ {A}_{t} , {\{ {X}_{m , 1 : T} \}}_{m = 1}^{M} , {\{ {Y}_{n , 1 : T} \}}_{n = 1}^{N} , {R}_{t} , {Q}_{t} , {θ}_{0 \vert 0} , {P}_{0 \vert 0}\end{matrix}$ |
| 2. | $Output : Projections {\{ {y}_{m , 1 : T}^{*} \}}_{m = 1}^{M}$ |
| 3. | Compute mean of source and target signals x̅_{1:T}, y̅_{1:T} |
| 4. | $\begin{matrix}Compute {\{ p ( {θ}_{t} \vert {\overline{x}}_{1 : t} , {\overline{y}}_{1 : t} , {x}_{m , 1 : t} ) \}}_{t = 1}^{T} \\ for \{ {\overline{x}}_{i : T} , {\overline{y}}_{1 : T} \} using Algorithm 1\end{matrix}$ |
| 5. | $Compute {\{ p ( {y}_{m , t}^{*} \vert {\overline{x}}_{1 : t} , {\overline{y}}_{1 : t} , {x}_{m , 1 : t} ) \}}_{t = 1}^{T} in Equation 39$ |
| 6. | for m = 1 to M do |
| 7. | for t = 1 to T do |
| 8. | $Compute Projections {y}_{m , t}^{*} = {c}_{m , t} {θ}_{t \vert t} as in Equation 40$ |
| 9. | $\begin{matrix}Compute projection variance \\ V ( {y}_{m , t}^{*} \vert {\overline{x}}_{1 : 𝔱} , {\overline{y}}_{1 : t} , {x}_{m , 1 : t} ) = {c}_{m , t} {P}_{t \vert t} {c}_{m , t}^{T} + {σ}^{2}\end{matrix}$ |
| 10. | end for |
| 11. | end for |

### Use Case 4: Predicting Missing Signal

The problem of predicting the profile of a parameter/variable at-scale by studying the behavior of the parameter/variable at other scales is now considered. Formally, this problem can be stated as follows: let y_{1,1:T} and y_{2,1:T} denote a variable (e.g. CO₂ flowrate) for Product A produced at Scale 1 (e.g., pilot-scale) and Scale 2 (e.g., commercial scale), respectively. Assuming that only y_{1,1:T} is known, the objective is to predict y_{2,1:T}. In other words, given the dynamics of a variable at Scale 1, the goal is to predict its dynamics at Scale 2. Note that in absence of a priori process knowledge or a clear understanding of the relationship between Scales 1 and 2, solving this problem, based on data alone, is nontrivial and can be quite difficult and cumbersome.

The application of the scaling method to predict y_{2,1:T} is discussed. First, an arbitrary product, Product B, was produced previously at Scales 1 and 2. Let x_{1,1:T} and x_{2,1:T} denote the dynamics of Product B at Scales 1 and 2, respectively. It is assumed that x_{1,1:T} and x_{2,1:T} are measured and available. Next, the AD ASTRA application 130 uses information from Product B to predict Product A at Scale 2. In some embodiments, this is done as follows: first, assume that the two signals y_{2,t} and x_{2,t}, are related according to the following scaling model:

$${y}_{2 , t} = [ 1 {x}_{2 , t} ] {θ}_{2 , t} + {e}_{2 , t}^{y} , \qquad ( Equation 41 )$$

where

$${\{ {e}_{2 , t}^{y} \}}_{t ∈ ℕ}$$

is a sequence of random variables distributed according to e_{2,t}~ [symbol] (⋅|0, Q_{2,t}). Using Algorithm 1, the scaling model generation unit 140 can calculate θ_{2,t} in Equation 41 as θ_{2,t}|(y_{2,1:t}, x_{2,1:t})~ [symbol] (⋅|θ_{2,t|t}, Σ_{2,t}). Now, given θ_{2,t|t} and x_{2,t}, the data conversion unit 142 can predict the signal y_{2,t} as follows:

$${\hat{y}}_{2 , t} = [ 1 {x}_{2 , t} ] {θ}_{2 , t \vert t} , \qquad ( Equation 42 )$$

where ŷ_{2,t} is a prediction of y_{2,t}. There are two important issues with the prediction in Equation 42. First, calculating θ_{2,t|t} using Algorithm 1 requires access to y_{2,1:t}, which is not available under the current problem setting, and second, the prediction in Equation 42 does not account for the uncertainty around the estimation of θ_{2,t}. Recall that while θ_{2,t}|(y_{2,1:t}, x_{2,1:t})~ [symbol] (⋅|θ_{2,t|t}, Σ_{2,t}), Equation 42 only uses the mean formation, θ_{2,t|t}, in Equation 42 to predict y_{2,t}. In this embodiment and use case, these issues are addressed using a Bayesian framework.

Given [symbol] ≡{x_{1,1:T}, y_{1,1:T}, x_{2,1:T}}, under the Bayesian framework, a posterior density

$${\{ p ( {y}_{2 , t} \vert {𝒢}_{1 : t} ) \}}_{t = 1}^{T}$$

is sought that encapsulates all the information available until time t to predict y_{2,t}. Using the law of marginalization, p(y_{2,t}| [symbol] ) can be alternatively written as

$$p ( {y}_{2 , t} \vert {𝒢}_{1 : t} ) = \int p ( {y}_{2 , t} , d {θ}_{2 , t} \vert {𝒢}_{1 : t} ) , \qquad ( Equation 43 )$$

where p(y_{2,t}, dθ_{2,t}| [symbol] ) is a joint distribution. Notably, the PDF p(y_{2,t}| [symbol] ) only depends on the observed data and not θ_{2,t}, which is both uncertain and unknown. Using the law of total probability and Markov property of Equations 42 and 43:

$$p ( {y}_{2 , t} \vert {𝒢}_{1 : t} ) = \int p ( {y}_{2 , t} \vert {θ}_{2 , t} , {x}_{2 , t} ) p ( d {θ}_{2 , t} \vert {y}_{1 , 1 : t} , {x}_{1 , 1 : t} ) , \qquad ( Equation 44 )$$

where p(y_{2,t}|θ_{2,t}, x_{2,t}) is a likelihood function, given by Equation 42 and p(dθ_{2,t}|y_{1,1:t}, x_{1,1:t}) is the conditional distribution for the scaling factor, θ_{2,t}, between the pair (y_{2,1:t}, x_{2,1:t}) given (y_{1,1:t}, x_{1,1:t}). Before proceeding further, two invariance hypotheses are defined: scale-invariance and product-invariance.

Referring first to scale-invariance, let p(θ_{1,t}|y_{1,1:t}, x_{1,1:t}) and p(θ_{2,t}|y_{2,1:t}, x_{2,1:t}) denote the posterior densities for the scaling factors between Products A and B at Scale 1 and Scale 2, respectively, where y_{1,1:T} and Y_{2,1:T} denote the data for Product A at Scales 1 and 2, respectively, and where x_{1,1:T} and x_{2,1:T} denote the data for Product B at Scales 1 and 2, respectively. The system is scale-invariant if the following relation holds for all t=1, . . . , T:

$$p ( {θ}_{1 , t} \vert {y}_{1 , 1 : t} , {x}_{1 , 1 : t} ) = p ( {θ}_{2 , t} \vert {y}_{2 , 1 : t} , {x}_{2 , 1 : t} ) \qquad ( Equation 45 )$$

Referring next to product-invariance, let p(θ_{3,t}|x_{2,1:t}, x_{1,1:t}) and p(θ_{4,t}|y_{2,1:t}, y_{1,1:t}) denote the posterior densities for the scaling factors for Product A and Product B between Scales 1 and 2, respectively, where y_{1,1:T} and y_{2,1:T} denote the data for Product A at Scales 1 and 2, respectively, and X_{1,1:T} and X_{2,1:T} denote the data for Product B at Scales 1 and 2, respectively. The system is product-invariant if the following relation holds for all t=1, . . . , T:

$$p ( {θ}_{3 , t} \vert {x}_{2 , 1 : t} , {x}_{1 , 1 : t} ) = p ( {θ}_{4 , t} \vert {y}_{2 , 1 : t} , {y}_{1 , 1 : t} ) \qquad ( Equation 46 )$$

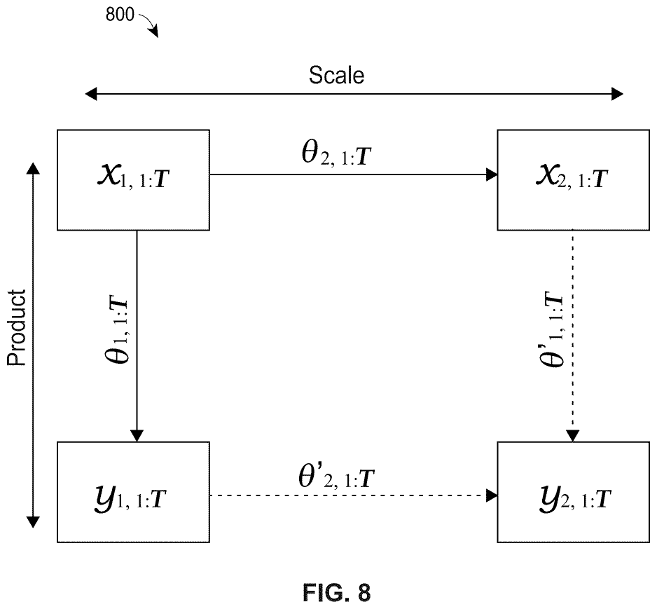

A schematic illustrating the scaling relationships 800 between Products A and B produced at Scales 1 and 2 is shown in FIG. 8. The solid rectangles in FIG. 8 represent variables that are measured (i.e., x_{1,1:T}, x_{2,1:T}, and y_{1,1:T}), and the dashed-line rectangle represents the variable that is missing (i.e., y_{2,1:T}). The scaling between different products at different scales is shown with arrows, with arrows pointing towards the target signals. The corresponding scaling factors are shown next to the arrows.

Under the current problem settings, Products A and B are assumed to be scale-invariant, i.e., the scaling between the products is preserved across different scales. Theoretically, scale-invariance is not a restrictive assumption since any similarities or dissimilarities between Products A and B at Scale 1 would continue to exist at Scale 2, as long as Products A and B are consistently produced (i.e., by maintaining initial conditions of processes across scales). In certain scenarios, the system may exhibit product-invariance, i.e., different products scale similarly across different scales. The method proposed in this section is also valid under the product-invariance hypothesis. Next, from the law of marginalization, p(θ_{2,t}|y_{1,1:t}, x_{1,1:t}) in Equation 44 can be written as follows:

$$\begin{matrix}p ( {θ}_{2 , t} \vert {y}_{1 , 1 : t} , {x}_{1 , 1 : t} ) = \int p ( {θ}_{2 , t} , {dy}_{2 , 1 : t} , {dx}_{2 , 1 : t} \vert {y}_{1 , 1 : t} , {x}_{1 , 1 : t} ) , & ( Equation 47 a ) \\ = \int p ( {θ}_{2 , t} \vert , {y}_{2 , 1 : t} , {x}_{2 , 1 : t} ) p ( {dy}_{2 , 1 : t} , {dx}_{2 , 1 : t} \vert {y}_{1 , 1 : t} , {x}_{1 , 1 : t} ) , & ( Equation 47 b ) \\ = p ( {θ}_{1 , t} \vert {y}_{1 , 1 : t} , {x}_{1 , 1 : t} ) \int p ( {dy}_{2 , 1 : t} , {dx}_{2 , 1 : t} \vert {y}_{1 , 1 : t} , {x}_{1 , 1 : t} ) , & ( Equation 47 c ) \\ = p ( {θ}_{1 , t} \vert {y}_{1 , 1 : t} , {x}_{1 , 1 : t} ) , & ( Equation 47 d )\end{matrix}$$

where from Equations 47b and 47c, the scale-invariance relation in Equation 45 is used. Next, substituting Equation 47d into Equation 44, one gets:

$$p ( {y}_{2 , t} \vert {𝒢}_{1 : t} ) = \int p ( {y}_{2 , t} \vert {θ}_{1 , t} , {x}_{2 , t} ) p ( d {θ}_{1 , t} \vert {y}_{1 , 1 : t} , {x}_{1 , 1 : t} ) , \qquad ( Equation 48 )$$

where p(y_{2,t}|θ_{1,t}, x_{2,t}) is a likelihood function for the model

$${y}_{2 , t} = [ 1 , {x}_{2 , t} ] {θ}_{1 , t} + {e}_{2 , t}^{y} , \qquad ( Equation 49 )$$

Comparing Equations 55 (below) and 41, it is clear that in the absence of true θ_{2,t} values, that value is replaced with θ_{1,t}, which is known a priori. As discussed in this section, the equivalency between θ_{1,t} and θ_{2,t} is established under the scale-invariance assumption in Equation 45.

$$p ( {y}_{2 , t} \vert {𝒢}_{1 : t} ) \approx \int p ( {y}_{2 , t} \vert {θ}_{1 , t} , {x}_{2 , t} ) \frac{1}{N} {\sum }_{i = 1}^{N} {δ}_{{θ}_{1 , t}^{( i )}} ( d {θ}_{1 , t )} , \qquad ( Equation 50 )$$

$$= \frac{1}{N} {\sum }_{i = 1}^{N} p ( {y}_{2 , t} \vert {θ}_{1 , t}^{( i )} , {x}_{2 , t} ) , \qquad ( Equation 51 )$$

$$p ( d {θ}_{1 , t} \vert {y}_{1 , 1 : t} , {x}_{1 , 1 : t} ) = \frac{1}{N} {\sum }_{i = 1}^{N} {δ}_{{θ}_{1 , t}^{( i )}} ( d {θ}_{1 , t} ) , \qquad ( Equation 52 )$$

y_{2,1:T} is predicted as follows: first, the scaling, θ_{1,1:T} is calculated between x_{1,1:T} and y_{1,1:T}, and then θ_{1,1:T} and x_{2,1:T} are used to predict y_{2,1:T}. Mathematically, let θ_{1,t}|(y_{1,1:t}, x_{1,1:t})~ [symbol] (θ_{1,t|t}, P_{1,t|t}) be the posterior for the scaling between y_{1,1:t} and x_{1,1:t} for all t=1, . . . , T, then an estimate of y_{1,t} is given as

$${\hat{y}}_{1 , t} = [ 1 , {x}_{1 , t} ] {θ}_{1 , t \vert t} , \qquad ( Equation 53 )$$

Similarly, if θ′_{1,t}|(y_{2,1:t}, x_{2,1:t})~ [symbol] (θ′_{1,t|t}, P′_{1,t|t}) is the scaling posterior between y_{2,1:t} and x_{2,1:t}, then y_{2,t} is estimated as

$${\hat{y}}_{2 , t} = [ 1 , {x}_{2 , t} ] {θ}_{1 , t \vert t}^{′} , \qquad ( Equation 54 )$$

While Equation 54 gives an optimal estimate of y_{2,t}, it is not very useful since θ′_{1,t|t} is unknown. However, under the scale-invariance assumption, θ′_{1,t|t} in Equation 54 can be replaced with θ_{1,t|t}, such that

$${\hat{y}}_{2 , t} = [ 1 , {x}_{2 , t} ] {θ}_{1 , t \vert t} , \qquad ( Equation 55 )$$

for all t=1, . . . , T. Under scale-invariance, the predictions in Equations 55 and 54 are not only optimal, but in fact the same, because θ_{1,t}|t=θ′_{1,t|t} for all t=1, . . . , T. The pseudo-code for predicting y_{2,1:T} using the proposed scaling method is given in Algorithm 4. Algorithm 4 is an offline method that predicts y_{2,1:T} even before Product A is produced at Scale 2. Further, that while the choice of Product B in Algorithm 4 is arbitrary, caution should be exercised to ensure it is scale-invariant to Product A.

The example Algorithm 4, which may be implemented by the AD ASTRA application 130, is as follows:

Algorithm 4 Predicting Missing Signal - Offline

| | |
|---|---|
| 1. | Input: Scaling model: Aₜ, X_{1,t}, Y_{1,t}, X_{2,t}, Rₜ, Qₜ, θ_{1,0\|0}, P_{1,0\|0} |
| 2. | Output: Predictions ŷ_{2\|t} |
| 3. | Initialize: θ_{1,0}~ [symbol] (·\|θ_{1,0\|0}, P_{1,0\|0}) |
| 4. | for t = 1 to T do |
| 5. | Set C_{1,t} ← [1, x_{1,t}]. Set C_{2,t} ← [1, x_{2,t}]. |
| 6. | Predict: θ_{1,t\|t-1} = Aₜθ_{1,t-1\|t-1} |
| 7. | $Predict \text{:} {P}_{1 , t \vert t - 1} = {Q}_{t - 1} + {A}_{𝔱} {θ}_{1 , t - 1 \vert t - 1} {A}_{t}^{T}$ |
| 8. | $Innovation : {S}_{t} = {C}_{1 , t} {P}_{1 , t \vert t - 1} {C}_{1 , t}^{T} + {R}_{t}$ |
| 9. | $Kalman gain : {K}_{t} = {P}_{1 , t \vert t - 1} {C}_{1 , t}^{T} {S}_{t}^{- 1}$ |
| 10. | Update: θ_{1,t\|t-1} = θ_{1,t\|t-1} + Kₜ(y_{1,t} − C_{1,t}θ_{1,t\|t-1}) |
| 11. | Update: P_{1,t\|t-1} = P_{1,t\|t-1} − KₜC_{1,t}P_{1,t\|t-1} |
| 12. | Predict: ŷ_{2,t} = C_{2,t}θ_{1,t\|t} |
| 13. | end for |

While the scale-invariance assumption in Algorithm 4 may not be restrictive in theory, it seldom holds in practice due to inherent process and raw-material variation and other known and unknown process disturbances. In other words, θ′_{1,t} may be significantly different from θ_{1,t}. This results in predictions with Equation 55 that may drift from the optimal predictions in Equation 54. To reduce such drifts, a real-time implementation of Algorithm 4 allows feedback of information from Product 1 at Scale 2 into Equation 55. Assuming y_{2,1:T} for k≤T is available the objective is to predict the remainder of the signal, y_{2,k+1:T}. As before, given y_{2,1:k}, the scaling posterior θ′_{1,t}(y_{2,1:t}, x_{2,1:t})~ [symbol] (θ′_{1,t|t}, P′_{1,t|t}) can be readily calculated using Algorithm 1. Now, for t≤k, ŷ_{2,t} can be re-predicted using Equation 54. However, for t>k, ŷ_{2,t} can be predicted as

$${\hat{y}}_{2 , t} = [ 1 , {x}_{2 , t} ] ( {θ}_{1 , t \vert t} + {δ}_{k} ) for t = k + 1 , … , T , \qquad ( Equation 56 )$$

where δₖ∈ [symbol] compensates for the differences between θ_{1,t|t} and θ′_{1,t|t}. For θₖΣ0_{2×1} the predictors of Equations 56 and 55 are the same. Now, since the only information available at t=k for Product A at Scale 2 is y_{2,1:k}, δₖ is defined as

$${δ}_{k} ≡ mean ( {\{ [ {θ}_{1 , t \vert t}^{′} - {θ}_{1 , t \vert t} ] \}}_{t = k - m}^{k} ) , \qquad ( Equation 57 )$$

where mean(⋅) is a mean function and m∈ [symbol] is a constant, such that k−m≥1. Physically, Equation 57 is the expected difference between θ_{1,t|t} and θ′_{1,t|t} at t=k−m, . . . , k. With Equation 57, the estimator of Equation 56 corrects future predictions based on the expected drift observed between θ_{1,t|t} and θ′_{1,t|t} in the past samples.

While δₖ in Equation 56 compensates for prediction drifts observed with Equation 55, it does not necessarily eliminate or prevent the predictor of Equation 55 from drifting in the first place. This is because of the inherent differences between θ_{1,t|t} and θ′_{1,t|t}. Recall that to estimate θ_{1,t|t}, Algorithm 1 only uses data set (x_{1,1:T}, y_{1,1:T}), and to estimate θ′_{1,t|t}, Algorithm 1 only uses (x_{2,1:t}, y_{2,1:t}). Now, to reduce the differences between θ_{1,t|t} and θ′_{1,t|t}, θ_{1,t|t} is projected closer to θ′_{1,t|t} by re-estimating θ_{1,t|t} by combining data sets (x_{1,1:T}, y_{1,1:T}) and (x_{2,1:k}, y_{2,1:k}) as follows:

$$\begin{matrix}{x}_{1 , 1 : T} ≡ [ {x}_{2 , 1 : k} , {x}_{1 , k + 1 : T} ] & {y}_{1 , 1 : T} ≡ [ {y}_{2 , 1 : k} , {y}_{1 , k + 1 : T} ] ,\end{matrix} \qquad ( Equation 58 )$$

for all k=1, . . . , T. Replacing a section of Scale 1 data with Scale 2 forces x_{1,1:T} and y_{1,1:T} in Equation 58 to become similar to x_{2,1:T} and y_{2,1:T}. In fact, for k=T, x_{1,1:T}=x_{2,1:T} and y_{1,1:T}=y_{2,1:T}. Now since Algorithm 1 estimates the posterior, p(θ_{1,t}|x_{2,1:t}, y_{2,1:t}) for all t=1, . . . , T using information available until time t, including Scale 2 data in Equation 58 ensures that θ_{1,t|t} and θ′_{1,t|t} are closer to each other. Notably, Equation 58 does not completely remove drifts with Equation 56, but rather only mitigates such drifts. Pseudo-code for the real-time prediction of y_{2,1:t} with the proposed scaling method is outlined in Algorithm 5. In Algorithm 5, δₖ is evaluated at each sampling time, but it can also be updated as needed.

The example Algorithm 5, which may be implemented by the AD ASTRA application 130, is as follows:

Algorithm 5 Predicting Missing Signal - Online

| | |
|---|---|
| 1. | Input: Scaling model: Aₜ, X_{1,t}, Y_{1,t}, X_{2,t}, Rₜ, Qₜ, θ_{1,0\|0}, P_{1,0\|0} |
| 2. | Output: Predictions ŷ_{2\|t} |
| 3. | Initialize: θ′_{1,0}~ [symbol] (·\|θ′_{1,0\|0}, P′_{1,0\|0}) |
| 4. | for t = 1 to T do |
| 5. | Set C_{2,t} ← [1, x_{2,t}] |
| 6. | Predict: θ′_{1,t\|t-1} = Aₜθ′_{1,t-1\|t-1} |
| 7. | $Predict \text{:} {P}_{1 , t \vert t - 1}^{′} = {Q}_{t - 1} + {A}_{t} {θ}_{1 , t - 1 \vert t - 1}^{′} {A}_{t}^{T}$ |
| 8. | $Innovation : {S}_{t} = {C}_{2 , t} {P}_{1 , t \vert t - 1}^{′} {C}_{2 , t}^{T} + {R}_{t}$ |
| 9. | $Kalman gain : {K}_{t}^{′} = {P}_{1 , t \vert t - 1}^{′} {C}_{2 , t}^{T} {S}_{t}^{- 1}$ |
| 10. | Update: θ′_{1,t\|t} = θ′_{1,t\|t-1} + K′ₜ(y_{2,t} − C_{2,t}θ′_{1,t\|t-1}) |
| 11. | Update: P′_{1,t\|t-1} = P′_{1,t\|t-1} − K′ₜC_{2,t}P′_{1,t\|t-1} |
| 12. | end for |
| 13. | Initialize: θ_{1,0}~ [symbol] (·\|θ_{1,0\|0}, P_{1,0\|0}) |
| 14. | for t = 1 to T do |
| 15. | if t ≤ k then |
| 16. | Set C_{1,t} ← [1, x_{2,t}] |
| 17. | Set y_{1,t} ← y_{2,t} |
| 18. | else |
| 19. | Set C_{1,t} ← [1, x_{1,t}] |
| 20. | end if |
| 21 | Predict: θ_{1,t\|t-1} = Aₜθ_{1,t-1\|t-1} |
| 22 | $Predict : {P}_{1 , t \vert t - 1} = {Q}_{t - 1} + {A}_{t} {θ}_{1 , t - 1 \vert t - 1} {A}_{t}^{T}$ |
| 23. | $Innovation : {S}_{t} = {C}_{1 , t} {P}_{1 , t \vert t - 1} {C}_{1 , t}^{T} + {R}_{t}$ |
| 24. | $Kalman gain : {K}_{t} = {P}_{1 , t \vert t - 1} {C}_{1 , t}^{T} {S}_{t}^{- 1}$ |
| 25. | Update: θ_{1,t\|t-1} = θ_{1,t\|t-1} + Kₜ(y_{1,t} − C_{1,t}θ_{1,t\|t-1}) |
| 26. | Update: P_{1,t\|t-1} = P_{1,t\|t-1} − KₜC_{1,t}P_{1,t\|t-1} |
| 27. | $Expected Drift : {δ}_{k} = mean ( {\{ {θ}_{1 , t \vert t} - {θ}_{1 , t \vert t}^{′} \}}_{j = k - m}^{k} )$ |
| 28. | Correction: \tilde{θ}_{1,t\|t} ← θ_{1,t\|t} + δₖ |
| 29. | if t ≤ k then |
| 30. | Predict: ŷ_{2\|t} = C_{1,t}θ_{1,t\|t} |
| 31. | else |
| 32. | Predict: ŷ_{2\|t} = C_{1,t}\tilde{θ}_{1,t\|t} |
| 33 | end if |
| 34. | end for |

### Use Case 4, Example A: Scale-Up of Monoclonal Antibody Production

The scale-up of a monoclonal antibody (say, Product A) production from a pilot-scale to a commercial-scale facility is now considered. During process scale-ups, it is routine to evaluate the automation, equipment, and operating constraints at the receiving/target site to ensure that the process can be successfully scaled-up and the product be produced at required specifications. For example, to account for increase in the production volume at the commercial scale, and to assess process parameters and constraints, several tasks, such as process characterization and gap analyses, are regularly performed. These studies lead to process and equipment changeover recommendations that typically need to be implemented before the product can be transferred and produced at the commercial facility.

In this example, a pilot-scale facility was fitted with a 300 liter fed-batch stainless steel bioreactor and a commercial facility ran a 15,000 liter fed-batch stainless steel bioreactor. To accommodate for the large production volume, the commercial bioreactor was operated at different aeration conditions than the pilot bioreactor. For example, the oxygen required to maintain the target dissolved oxygen is much higher for the commercial bioreactor than for the pilot bioreactor. To ensure that the pumps at the commercial facility are able to supply the required oxygen, and at the required rate, it is critical to first predict what the oxygen demands at the commercial scale would be. Instead of predicting oxygen demands at each sampling time, which may help better assess the power ratings for the pumps and other key attributes, the current practice is to only predict peak oxygen demand using simple volumetric scaling methods. The predictions based on volumetric scaling methods are only approximate at best, as these methods do not take into account process disturbances or specific process configurations that may affect the actual oxygen demand in the commercial bioreactor. For example, if the commercial bioreactor is fitted with a less efficient impeller design, then the actual oxygen required to maintain the target dissolved oxygen levels would be different from that suggested by the volumetric scaling method.

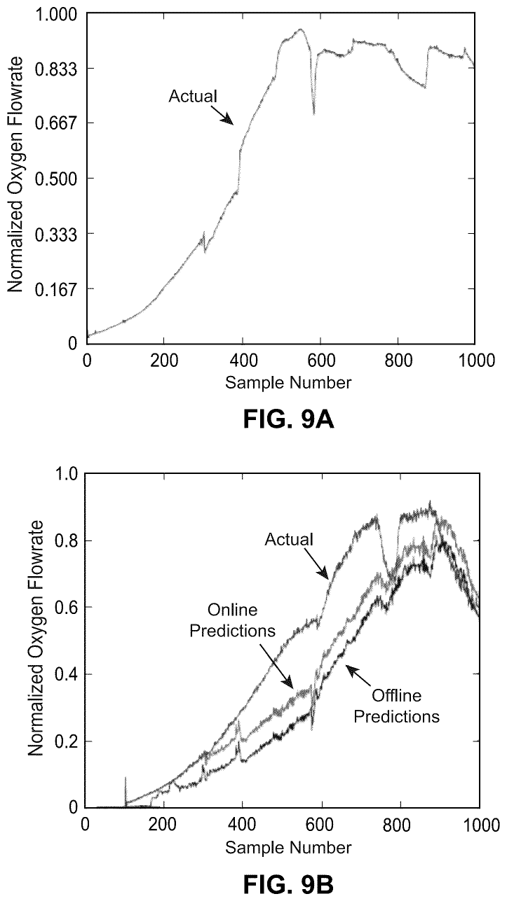

Here, the scaling method discussed above for predicting a missing signal was used to predict the oxygen demand in the commercial bioreactor at each sampling time. FIG. 9A gives the normalized oxygen demand for the Product A in the pilot bioreactor. As seen in FIG. 9A, as the cells grow, the oxygen required to maintain the target dissolved oxygen levels also increases. Next, an arbitrary product, Product B, was introduced, where Product B was previously produced both at the pilot-scale and the commercial-scale facilities. For the sake of brevity, the oxygen demand profiles for Product B in the pilot and commercial bioreactors are not shown. Now, given the oxygen profiles for Product A at pilot-scale and Product B at pilot and commercial scales, an application corresponding to an embodiment of the AD ASTRA application 130 used Algorithm 3 to predict oxygen demands for Product A at the commercial scale. FIG. 9B compares “offline” predictions from Algorithm 3 against the “actual” oxygen demand (both normalized). As seen in FIG. 9B, Algorithm 3 predicts oxygen demand at each sampling time, including at peak conditions that also correspond to the maximum VCD. While there is an offset between the offline predicted, ŷ_{2,t}, and actual, y_{2,t}, the overall trends are in close agreement. The offset between ŷ_{2,t} and y_{2,t} can be calculated as

$$ε = \frac{1}{T} {\sum }_{t = 1}^{T} {( {\hat{y}}_{2 , t} - {y}_{2 , t} )}^{2} , \qquad ( Equation 57 )$$

where E is the mean-square error (MSE). The MSE for Algorithm 3 in FIG. 9B is 625.97. There could be several reasons for this high MSE for Algorithm 3. As discussed earlier, the scale-invariance assumption in Algorithm 3 may not be entirely valid for this particular scale-up study. In FIG. 9B, the offset between sample numbers 200 and 900 is far greater than for samples in the 1 to 200 and 900 to 1000 ranges. This suggests that the scale-invariance assumption for oxygen demand is valid mostly at the start-up and shut-down phases of the bioreactor.

The normalized values of the peak oxygen demand (as a normalized flow rate) predicted by the volumetric scaling method and Algorithm 3 are 0.691 and 0.813, respectively, against the actual peak demand at 0.918. Clearly, the prediction from Algorithm 3 is much more accurate compared to the prediction from the volumetric method. This further demonstrates the efficacy of Algorithm 3 in predicting oxygen demand in the bioreactor.

Next, to mitigate the offset observed in FIG. 9B for the “offline” prediction of Algorithm 3, Algorithm 4 was used. As discussed, Algorithm 4 is an “online” method that uses information from the at-scale bioreactor to improve future predictions. FIG. 9B also compares the online predictions from Algorithm 4 against the actual demand. The results in FIG. 9B are presented for t=300, which means that y_{2,1:300} is assumed to be available. It is not surprising that the y_{2,1:300} with Algorithm 4 is close to y_{2,1:300}, since y_{2,1:300} is already known. Overall, the online predictions with Algorithm 4 are much closer to the actual oxygen demand as compared to the offline predictions with Algorithm 3. The MSE with Algorithm 4 for t=301, . . . , 1000 is 0.889 (normalized) compared to the MSE of 1.738 (normalized) with Algorithm 3 in the same interval. This clearly demonstrates the improvement Algorithm 4 is able to achieve over Algorithm 3. Finally, the peak oxygen demand (as a normalized flow rate) predicted by Algorithm 4 is 0.874, which is much closer to the actual oxygen demand of 0.918 than the peak demand of 0.813 predicted by Algorithm 3. This again demonstrates the efficacy of Algorithm 4 in yielding improved predictions over Algorithm 3. However, both Algorithm 3 and Algorithm 4 provide significant improvements in predicting oxygen demand in the commercial bioreactor over current methods used in the biopharmaceutical industry.

Additional considerations pertaining to this disclosure will now be addressed.

Some of the figures described herein illustrate example block diagrams having one or more functional components. It will be understood that such block diagrams are for illustrative purposes and the devices described and shown may have additional, fewer, or alternate components than those illustrated. Additionally, in various embodiments, the components (as well as the functionality provided by the respective components) may be associated with or otherwise integrated as part of any suitable components.

Embodiments of the disclosure relate to a non-transitory computer-readable storage medium having computer code thereon for performing various computer-implemented operations. The term “computer-readable storage medium” is used herein to include any medium that is capable of storing or encoding a sequence of instructions or computer codes for performing the operations, methodologies, and techniques described herein. The media and computer code may be those specially designed and constructed for the purposes of the embodiments of the disclosure, or they may be of the kind well known and available to those having skill in the computer software arts. Examples of computer-readable storage media include, but are not limited to: magnetic media such as hard disks, floppy disks, and magnetic tape; optical media such as CD-ROMs and holographic devices; magneto-optical media such as optical disks; and hardware devices that are specially configured to store and execute program code, such as ASICs, programmable logic devices (“PLDs”), and ROM and RAM devices.

Examples of computer code include machine code, such as produced by a compiler, and files containing higher-level code that are executed by a computer using an interpreter or a compiler. For example, an embodiment of the disclosure may be implemented using Java, C++, or other object-oriented programming language and development tools. Additional examples of computer code include encrypted code and compressed code. Moreover, an embodiment of the disclosure may be downloaded as a computer program product, which may be transferred from a remote computer (e.g., a server computer) to a requesting computer (e.g., a client computer or a different server computer) via a transmission channel. Another embodiment of the disclosure may be implemented in hardwired circuitry in place of, or in combination with, machine-executable software instructions.

As used herein, the singular terms “a,” “an,” and “the” may include plural referents, unless the context clearly dictates otherwise.

As used herein, the terms “approximately,” “substantially,” “substantial” and “about” are used to describe and account for small variations. When used in conjunction with an event or circumstance, the terms can refer to instances in which the event or circumstance occurs precisely as well as instances in which the event or circumstance occurs to a close approximation. For example, when used in conjunction with a numerical value, the terms can refer to a range of variation less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. For example, two numerical values can be deemed to be “substantially” the same if a difference between the values is less than or equal to ±10% of an average of the values, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%.

Additionally, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified.

While the present disclosure has been described and illustrated with reference to specific embodiments thereof, these descriptions and illustrations do not limit the present disclosure. It should be understood by those skilled in the art that various changes may be made and equivalents may be substituted without departing from the true spirit and scope of the present disclosure as defined by the appended claims. The illustrations may not be necessarily drawn to scale. There may be distinctions between the artistic renditions in the present disclosure and the actual apparatus due to manufacturing processes, tolerances and/or other reasons. There may be other embodiments of the present disclosure which are not specifically illustrated. The specification (other than the claims) and drawings are to be regarded as illustrative rather than restrictive. Modifications may be made to adapt a particular situation, material, composition of matter, technique, or process to the objective, spirit and scope of the present disclosure. All such modifications are intended to be within the scope of the claims appended hereto. While the techniques disclosed herein have been described with reference to particular operations performed in a particular order, it will be understood that these operations may be combined, sub-divided, or re-ordered to form an equivalent technique without departing from the teachings of the present disclosure. Accordingly, unless specifically indicated herein, the order and grouping of the operations are not limitations of the present disclosure.

1. A method for scaling data across different processes, the method comprising:
obtaining first time-series data indicative of one or more input, state, and/or output parameters of a first process over time;
obtaining second time-series data indicative of one or more input, state, and/or output parameters of a second process over time;
generating, by one or more processors, a scaling model specifying time-varying scaling relationships between the input, state, and/or output parameters of the first process and the input, state, and/or output parameters of the second process;
transferring, by the one or more processors and using the scaling model, source time-series data associated with a source process to target time-series data associated with a target process, wherein the source time-series data is indicative of one or more input, state, and/or output parameters of the source process over time, and wherein the target time-series data is indicative of one or more input, state, and/or output parameters of the target process over time; and
storing, by the one or more processors, the target time-series data in memory.

2. The method of claim 1, wherein the scaling model comprises a probabilistic estimator.

3. The method of claim 2, wherein the probabilistic estimator is a Kalman filter.

4. The method of claim 1, wherein the first process is the source process and the second process is the target process.

5. The method of claim 1, wherein the first process and the source process are associated with a first process site, and wherein the second process and the target process are associated with a second process site different than the first process site.

6. The method of claim 1, wherein the first process and the source process are associated with a first process scale, and the second process and the target process are associated with a second process scale different than the first process scale.

7. The method of claim 6, wherein the first process and the source process are bioreactor processes using a first bioreactor size, and the second process and the target process are bioreactor processes using a second bioreactor size, the first bioreactor size being smaller than the second bioreactor size.

8. The method of claim 1, further comprising:
training, by the one or more processors, a machine learning model of the target process using the target time-series data; and
controlling one or more inputs to the target process using the trained machine learning model.

9. The method of claim 1, wherein:
the first process, the second process, the source process, and the target process are bioreactor processes; and
the input, state, and/or output parameters of the first process, the second process, the source process, and the target process include oxygen flow rate, pH, agitation, and/or dissolved oxygen.

10. The method of claim 1, wherein the first process and the source process are bioreactor processes in which a first biopharmaceutical product grows, and the second process and the target process are bioreactor processes in which a second biopharmaceutical product different than the first biopharmaceutical product grows.

11. The method of claim 1, further comprising, before transferring the source time-series data:
obtaining, by the one or more processors, additional time-series data indicative of one or more input, state, and/or output parameters of one or more additional processes over time;
generating, by the one or more processors, one or more additional scaling models each specifying time-varying relationships between the input, state, and/or output parameters of the first process and the input, state, and/or output parameters of a respective one of the one or more additional processes; and
determining, by the one or more processors and based on the scaling model and the one or more additional scaling models, that the input, state, and/or output parameters of the second process have a closest measure of similarity to the input, state, and/or output parameters of the first process.

12. The method of claim 11, wherein determining that the input, state, and/or output parameters of the second process have the closest measure of similarity to the input, state, and/or output parameters of the first process includes using a Kullback-Leibler divergence (KLD) measure of similarity or Weitzman's measure of similarity.

13. The method of claim 1, wherein:
the first process is a first bioreactor process at a first process scale;
the second process is a second bioreactor process at a second process scale;
the source process is a third bioreactor process at the first process scale;
the target process is a fourth bioreactor process at the second process scale;
the first, second, third, and fourth bioreactor processes are different processes; and
the first process scale is different than the second process scale.

14. The method of claim 1, wherein at least a portion of transferring the source time-series data to the target time-series data associated occurs substantially in real-time as the source time-series data is obtained.

15. The method of claim 1, further comprising:
providing, by the one or more processors and via a display device, a user interface to a user; and
receiving, by the one or more processors and from the user via the user interface, a control setting,
wherein generating the scaling model includes using the control setting to set a covariance when generating the scaling model.

16. A system comprising:
one or more processors; and
one or more computer-readable media storing instructions that, when executed by the one or more processors, cause the one or more processors to:
obtain first time-series data indicative of one or more input, state, and/or output parameters of a first process over time,
obtain second time-series data indicative of one or more input, state, and/or output parameters of a second process over time,
generate a scaling model specifying time-varying scaling relationships between the input, state, and/or output parameters of the first process and the input, state, and/or output parameters of the second process,
transfer, using the scaling model, source time-series data associated with a source process to target time-series data associated with a target process, wherein the source time-series data is indicative of one or more input, state, and/or output parameters of the source process over time, and wherein the target time-series data is indicative of one or more input, state, and/or output parameters of the target process over time; and
store the target time-series data in memory.

17. The system of claim 16, wherein the scaling model comprises a probabilistic estimator.

18. The system of claim 17, wherein the probabilistic estimator is a Kalman filter.

19. The system of claim 16, wherein the first process is the source process and the second process is the target process.

20. The system of claim 16, wherein the first process and the source process are associated with a first process site, and wherein the second process and the target process are associated with a second process site different than the first process site.

21. The system of claim 16, wherein the first process and the source process are associated with a first process scale, and the second process and the target process are associated with a second process scale different than the first process scale.

22. The system of claim 21, wherein the first process and the source process are bioreactor processes using a first bioreactor size, and the second process and the target process are bioreactor processes using a second bioreactor size, the first bioreactor size being smaller than the second bioreactor size.

23. The system of claim 16, wherein:
the instructions further cause the one or more processors to train a machine learning model of the target process using the target time-series data; and
the system further comprises one or more controllers configured to control one or more inputs to the target process using the trained machine learning model.

24. The system of claim 16, wherein:
the first process, the second process, the source process, and the target process are bioreactor processes; and
the input, state, and/or output parameters of the first process, the second process, the source process, and the target process include oxygen flow rate, pH, agitation, and/or dissolved oxygen.

25. The system of claim 16, wherein the first process and the source process are bioreactor processes in which a first biopharmaceutical product grows, and the second process and the target process are bioreactor processes in which a second biopharmaceutical product different than the first biopharmaceutical product grows.

26. The system of claim 16, wherein the instructions further cause the one or more processors to, before transferring the source time-series data:
obtain additional time-series data indicative of one or more input, state, and/or output parameters of one or more additional processes over time;
generate one or more additional scaling models each specifying scaling time-varying relationships between the input, state, and/or output parameters of the first process and the input, state, and/or output parameters of a respective one of the one or more additional processes; and
determine, based on the scaling model and the one or more additional scaling models, that the input, state, and/or output parameters of the second process have a closest measure of similarity to the input, state, and/or output parameters of the first process.

27. The system of claim 26, wherein determining that the input, state, and/or output parameters of the second process have the closest measure of similarity to the input, state, and/or output parameters of the first process includes using a Kullback-Leibler divergence (KLD) measure of similarity or Weitzman's measure of similarity.

28. The system of claim 16, wherein:
the first process is a first bioreactor process at a first process scale;
the second process is a second bioreactor process at a second process scale;
the source process is a third bioreactor process at the first process scale;
the target process is a fourth bioreactor process at the second process scale;
the first, second, third, and fourth bioreactor processes are different processes; and
the first process scale is different than the second process scale.

29. The system of claim 16, wherein at least a portion of transferring the source time-series data to the target time-series data associated occurs substantially in real-time as the source time-series data is obtained.

30. The system of claim 16, further comprising a display device, and wherein the instructions further cause the one or more processors to:
provide, via the display device, a user interface to a user; and
receive, from the user via the user interface, a control setting,
wherein generating the scaling model includes using the control setting to set a covariance when generating the scaling model.
